(19) United States
(12) Patent Application Publication
(10) Pub. No.: US 2024/0182974 A1
(43) Pub. Date: Jun. 6, 2024

(54) IMMUNE CELL QUANTIFICATION

(71) Applicant: ACADEMISCH ZIEKENHUIS LEIDEN (H.O.D.N. LUMC), Leiden (NL)

(72) Inventors: Willem H. ZOUTMAN, Alphen aan den Rijn (NL); Pieter A. VAN DER VELDEN, Leiden (NL); Rogier J. NELL, Voorschoten (NL)

(21) Appl. No.: 17/767,788

(22) PCT Filed: Oct. 8, 2020
(86) PCT No.: PCT/NL2020/050622

§ 371 Date: Apr. 8, 2022

(30) Foreign Application Priority Data
Oct. 9, 2019 (NL) 2023987

(51) Int. Cl. C12Q 1/6886; C12Q 1/686; C12Q 1/6881

(52) U.S. Cl. CPC C12Q 1/6886; C12Q 1/686; C12Q 1/6881; C12Q 2600/118

(57) ABSTRACT

The present invention provides novel methods and kits for quantifying immune cells, specifically B-cells and T-cells, in a sample. The methods and kits may be used to monitor disease progression. They may also be used to determine the effect of a medicament used in the treatment of a disease. They may also be used to determine disease prognosis. They may also be used to diagnose disease.

The present invention provides novel methods and kits for quantifying immune cells, specifically B-cells and T-cells, in a sample. The methods and kits may be used to monitor disease progression, determine the effect of a medicament used in the treatment of a disease, determine disease prognosis, and/or diagnose disease.

### BACKGROUND

T-cells play an important role in cell-mediated immunity. Quantifying T-cells accurately in benign, inflammatory and malignant tissues or body fluids is of great importance in a variety of clinical applications. For instance, quantifying T-cells in benign or (chronic) inflammatory diseases can be valuable in terms of diagnostics. With respect to malignancies, the magnitude of T-cell infiltration has been correlated positively (and negatively) to tumour growth and clinical prognosis. Moreover, the extent of T-cell migration can serve as a predictive factor for expected response to neoadjuvant therapies. Furthermore, (infiltrated) T-cells are being increasingly used therapeutically by the administration of checkpoint inhibitors. Accurate quantification of (infiltrated) T-cells is therefore valuable and of great importance in the clinic.

The conventional quantification methods for determining T-cell content in body fluids or in solid tissues are flow cytometry and immunohistochemistry, respectively. Both methods use T-cell-specific antibodies and therefore are very precise. However, they require that the requisite T-cell markers and epitopes are present and accessible in the test sample. The presence and accessibility of epitopes depends on the specimen's condition and the preparation method that is used. In general, fresh, frozen and fixed materials can meet the required criteria for accurate quantification of T-cells (Walker, 2006; Wood et al., 2013). However, when sample quantity and/or quality is too low, quantification can be impeded, and the focus must be shifted from epitopes to T-cell-specific DNA biomarkers.

Moreover, generic T-cell epitopes may vary in expression between different T-cell populations. For example, T-cell receptors in healthy individuals can gradually be expressed at lower levels in the elderly. Furthermore, it is known that differential expression of these specific cell markers frequently takes place in T-cell malignancies. Accurate T-cell quantification using T-cell epitopes may therefore be adversely affected by heterogeneous T-cell epitope expression within the population. By contrast, genomic DNA is typically present in equal (diploid) amounts per cell. Hence, the concentration of DNA molecules in a sample is generally a more accurate reflection of the number of cells in the sample. In other words, T-cell DNA markers generally represent the number of T-cells in a sample more accurately than corresponding transcriptionally and translationally expressed molecular markers.

T-cell receptors (TCRs) are translationally expressed on mature T-cells as heterodimer receptors. In peripheral blood, the majority of T-cells possesses αβTCRs, while a smaller fraction (1-5%) consists of γδ T-cells. Mature T-cells differ genetically from other cell types as a result of TCR gene rearrangements. During early lymphoid differentiation, many distinct variable (V), diversity (D) and joining (J) TCR gene segments are rearranged. This specific type of programmed genetic recombination forms the basis of the combinatorial diversity of TCR molecules (Davis and Bjorkman, 1988). Ultimately, VDJ gene rearrangements, followed by DNA sequence altering mechanisms like junctional diversity and completing combinatorial association of the translated TCR chains, result in a highly diverse repertoire of antigenic TCRs. Four gene complexes are responsible for the variety of expressed TCRs and rearrange sequentially in a highly ordered manner (including allelic exclusion), starting with TRD, followed by TRG, TRB, and finally TRA. Since the process of TCR rearrangements is extremely error-prone, the cascade of sequentially executed rearrangements continues from TRD to TRA until a functional recombined TCR sequence is obtained. A functional TCR is a heterodimer receptor and is encoded by either a functional rearranged TRG and TRD allele (γδTCRs) or TRA and TRB allele (αβTCRs). However, some parts of the four TCR genes rearrange biallelically regardless of the order of recombination and become deleted early in T-cell maturation (Dik et al., 2005). For instance, sequences located in the intergenic regions Dδ2-Dδ3 (TRD gene at 14q11.2) and Dβ1-Jβ1.1 (TRB gene at 7q34) are lost in mature T-cells (these regions are referred to as ΔD and ΔB, respectively herein). Since these specific TCR loci are lost in mature T-cells, they can be considered as genomic biomarkers for this cell type. By measuring loss of ΔD and/or ΔB germline TCR loci in DNA specimens, it is possible to determine the fraction of T-cells in a mixed cell sample in a quantitative manner (Zoutman et al., 2017).

Parallel TCR analyses have been performed in order to identify T-cell receptor re-arrangements. However, these analyses do not provide a T-cell quantification (Pongers-Willemse et al., 1999). Conventionally, multiplex PCR, combined with deep sequencing techniques, can be applied to determine T-cell content on a genomic level. However, these approaches typically require an amplification step, thereby limiting possibilities for absolute quantification and allowing merely for interpretation of relative differences. Moreover, these approaches target the whole repertoire of the T-cell receptor genes and thereby supply additional information about gene use (van Dongen et al., 2003). Consequently, a simple T-cell quantification results into a complex, expensive and time-consuming procedure. B-cells play an important role in adaptive cell-mediated and humoral immunity. Hence, quantifying B-cells accurately in benign, inflammatory and malignant tissues or body fluids can also be of great importance in the clinic. For instance, quantifying B-cells in benign and (chronic) inflammatory diseases can be valuable in terms of diagnostics. In autoimmune diseases like arthritis the fraction of B-cells is commonly ascertained as a means monitoring disease progression. Furthermore, since directed B-cell eradication is one of the treatment modalities for inflammatory diseases, monitoring treatment efficacy by accurate B-cell quantification is also warranted.

With respect to malignancies, although the magnitude of T-cell infiltration has been correlated both positively and negatively to tumour growth and clinical prognosis, the role of B-cells is underestimated. Increasing evidence supports a correlation between B-cell infiltration and clinical prognosis and prediction to therapy response. Furthermore, some studies associate B-cell infiltration with an impaired immune response. Eradication of the B-cell compartment has also been suggested as a therapy to improve anti-tumour response. Hence, accurate quantification of B-cells is also valuable and of great importance in the clinic.

There is a need for new methods for accurately quantifying B-cells and T-cells in a DNA sample independent of cellular context.

### BRIEF SUMMARY OF THE DISCLOSURE

The inventors have developed new DNA-based methods for accurate B- and T-cell quantification. The new methods are based on structural changes at the DNA level that are unique to B- and T-cells. The inventors have also shown that these methods can advantageously be adapted to distinguish between switched and non-switched B-cells. The methods can advantageously be used in several settings, including monitoring B- and/or T-cell number (and/or purity) in clinical procedures. For example, during CAR-T cell therapy, the methods described herein may be used to monitor the T-cell number (and/or purity) throughout the procedure. Besides this clinical application there are also numerous scientific procedures in which it would be advantageous to monitor B- and/or T-cell number (and/or purity).

A method is provided for determining the VDJ rearranged human T-cell fraction in a sample, the method comprising:
a) quantifying, in the sample, the amount of:
   a diploid reference DNA marker;
a TCR DNA marker selected from an intergenic region between Dδ2 and Dδ3 on chromosome 14q11.2 or an intergenic region between Dβ1 and Jβ1.1 on chromosome 7q34; and
   a DNA regional corrector of the TCR marker; and
b) determining the VDJ rearranged human T-cell fraction in the sample based on the quantification obtained in step a).

Suitably, the VDJ rearranged human T-cells may express a T-cell receptor.

Suitably, the VDJ rearranged T-cell fraction may be determined as:

T-cell fraction=([DNA regional corrector]−[TCR DNA marker])/[diploid reference DNA marker].

Suitably, the TCR DNA marker may be an intergenic region between Dδ2 and Dδ3 on chromosome 14q11.2 and the DNA regional corrector may be selected from the group consisting of: CHD8, METTL3, SALL2 and TOX4.

Suitably, the TCR DNA marker may be an intergenic region between Dβ1 and Jβ1.1 on chromosome 7q34 and the DNA regional corrector may be selected from the group consisting of: TRBC2, BRAF, MOXD2P, PRSS58, MGAM, TAS2R38, and CLEC5A.

Suitably, the diploid reference DNA marker may be selected from the group consisting of: exon 14 of DNM3, TTC5, TERT, VOPP1.

Suitably, the sample may comprise malignant cells and/or cells with DNA copy number instability.

Suitably, the sample may originate from malignant cells and/or the sample may originate from cells with DNA copy number instability.

Suitably, the sample may comprise DNA having copy number alterations of chromosome 14q or chromosome 7q.

Suitably, the sample may be a tissue sample or a body fluid sample, optionally wherein the body fluid sample is vitreous fluid, cerebrospinal fluid, peritoneal fluid, amniotic fluid, pleural fluid or synovial fluid.

Suitably, the diploid reference DNA marker, TCR DNA marker and DNA regional corrector may be quantified using a multiplex assay.

Suitably, the diploid reference DNA marker, TCR DNA marker and regional corrector may be quantified by digital PCR.

Suitably, the sample may be obtained from a subject.

Suitably, the method may be for monitoring disease progression, determining the effect of a medicament used in the treatment of a disease, determining disease prognosis, or diagnosing a disease.

Suitably, the disease may be an infectious disease, an autoimmune disease or a cancer.

Suitably, the cancer may be uveal melanoma, skin melanoma or any other solid tumour.

Suitably, the autoimmune disease may be rheumatoid arthritis, multiple sclerosis, type 1 diabetes or inflammatory bowel disease.

Suitably, the infectious disease may be:
(i) a viral infection, optionally wherein the viral infection is HIV or hepatitis; or
(ii) a bacterial infection, optionally wherein the bacterial infection is tuberculosis or pertussis.

A method is also provided for determining the VDJ rearranged human B-cell fraction in a sample, the method comprising:
a) quantifying, in the sample, the amount of:
   a diploid reference DNA marker; and
a B-cell DNA marker comprising an intergenic sequence between IGHD7-27 and IGHJ1 at chromosome 14q32.33; and
b) determining the VDJ rearranged human B-cell fraction in the sample based on the quantification obtained in step a).

Suitably, the VDJ rearranged human B-cell fraction may be determined as:

B-cell fraction=1−([B-cell DNA marker]/[diploid reference DNA marker]).

Suitably, step a) of the method may further comprise quantifying, in the sample, a DNA regional corrector of the B-cell DNA marker and determining the VDJ rearranged human B-cell fraction as:

B-cell fraction=([DNA regional corrector]−[B-cell DNA marker])/[diploid reference DNA marker].

Suitably, the regional corrector may be selected from the group consisting of: IGHA2, TMEM121, MARK3, BAG5, KLC1, MTA1, CRIP2, PACS2, BRF1, JAG2 and PLD4.

Suitably, step a) of the method further may comprise determining the class-switched VDJ rearranged human B-cell fraction in the sample by:
i) quantifying, in the sample, a class-switched B-cell DNA marker comprising a sequence of IGHD at chromosome 14q32.33; and
ii) determining the class-switched VDJ rearranged human B-cell fraction.

A method is also provided for determining the class-switched human B-cell fraction in a sample, the method comprising:
a) quantifying, in the sample, the amount of:
   a diploid reference DNA marker; and
a class-switched B-cell DNA marker comprising a sequence of IGHD at chromosome 14q32.33; and
b) determining the class-switched human B-cell fraction in the sample based on the quantification obtained in step a).

Suitably, the class-switched human B-cell fraction may be determined as:

class-switched fraction=1−([class-switched B-cell DNA marker]/[diploid reference DNA marker]).

Suitably, the class-switched human B-cell fraction may be determined as:

class-switched fraction={1−([class-switched B-cell DNA marker]/[diploid reference DNA marker])}/allelic factor for the class-switched B-cell DNA marker.

Suitably, step a) of the method may further comprise quantifying, in the sample, a DNA regional corrector of the class-switched B-cell DNA marker and determining the class-switched human B-cell fraction as:

class-switched fraction=([DNA regional corrector]−[class-switched B-cell DNA marker])/[diploid reference DNA marker].

Suitably, the class-switched human B-cell fraction may be determined as:

class-switched fraction={([DNA regional corrector]−[class-switched B-cell DNA marker])/[diploid reference DNA marker]}/allelic factor for the class-switched B-cell DNA marker.

Suitably, the regional corrector may be selected from the group consisting of: IGHA2, TMEM121, MARK3, BAG5, KLC1, MTA1, CRIP2, PACS2, BRF1, JAG2 and PLD4.

Suitably, the VDJ rearranged human B-cells may express a B-cell receptor or an antibody.

Suitably, the diploid reference DNA marker may be selected from the group consisting of: exon 14 of DNM3, TTC5, TERT, VOPP1.

Suitably, the sample may comprise malignant cells and/or cells with DNA copy number instability.

Suitably, the sample may originate from malignant cells and/or the sample may originate from cells with DNA copy number instability.

Suitably, the sample may comprise DNA having copy number alterations of chromosome 14q.

Suitably, the sample may be a tissue sample or a body fluid sample, optionally wherein the body fluid sample is vitreous fluid, cerebrospinal fluid, peritoneal fluid, amniotic fluid, pleural fluid or synovial fluid.

Suitably, the diploid reference DNA marker, B-cell DNA marker and optionally the DNA regional corrector may be quantified using a multiplex assay.

Suitably, the diploid reference DNA marker, B-cell DNA marker and optionally the DNA regional corrector may be quantified by digital PCR.

Suitably, the sample may be obtained from a subject.

Suitably, the method may be for monitoring disease progression, determining the effect of a medicament used in the treatment of a disease, determining disease prognosis, or diagnosing a disease.

Suitably, the disease may be selected from an infectious disease, an autoimmune disease or a cancer.

Suitably, the cancer may be a B-cell lymphoma or any solid tumour that becomes inflamed, optionally wherein the solid tumour is melanoma.

Suitably, the autoimmune disease may be rheumatoid arthritis, multiple sclerosis, type 1 diabetes or inflammatory bowel disease.

Suitably, the infectious disease may be:
(i) a viral infection, optionally wherein the viral infection is hepatitis; or
(ii) a bacterial infection, optionally wherein the bacterial infection is tuberculosis or pertussis.

A kit is also provided for determining the VDJ rearranged human T-cell fraction in a sample, the kit comprising:
a) at least one primer (e.g. at least two primers) and/or a probe for specifically amplifying a diploid reference DNA marker;
b) at least one primer (e.g. at least two primers) and/or a probe for specifically amplifying a TCR DNA marker selected from an intergenic region between Dδ2 and Dδ3 on chromosome 14q11.2 or an intergenic region between Dβ1 and Jβ1.1 on chromosome 7q34; and
c) at least one primer (e.g. at least two primers) and/or a probe for specifically amplifying a 15 DNA regional corrector of the TCR marker.

Suitably,
(i) the TCR DNA marker may be an intergenic region between D52 and D53 on chromosome 14q11.2 and the DNA regional corrector may be selected from the group consisting of: CHD8, METTL3, SALL2 and TOX4; or
(ii) the TCR DNA marker may be an intergenic region between Dδ1 and Jβ1.1 on chromosome 7q34 and the DNA regional corrector may be selected from the group consisting of: TRBC2, BRAF, MOXD2P, PRSS58, MGAM, TAS2R38, and CLEC5A.

Suitably, the diploid reference DNA marker may be selected from the group consisting of: exon 14 of DNM3, TTC5, TERT, VOPP1.

A kit is also provided for determining the VDJ rearranged human B-cell fraction in a sample, the kit comprising:
a) at least one primer (e.g. at least two primers) and/or a probe for specifically amplifying a diploid reference DNA marker; and
b) at least one primer (e.g. at least two primers) and/or a probe for specifically amplifying a B-cell DNA marker comprising an intergenic sequence between IGHD7-27 and IGHJ1 at chromosome 14q32.33;
and optionally at least one primer (e.g. at least two primers) and/or a probe for specifically amplifying a DNA regional corrector of the B-cell DNA marker.

Suitably, the kit may further comprise:
c) at least one primer (e.g. at least two primers) and/or a probe for specifically amplifying a class-switched B-cell DNA marker comprising a sequence of IGHD at chromosome 14q32.33.

A kit is also provided for determining the class-switched human B-cell fraction in a sample, the kit comprising:
a) at least one primer (e.g. at least two primers) and/or a probe for specifically amplifying a diploid reference DNA marker; and
b) at least one primer (e.g. at least two primers) and/or a probe for specifically amplifying a class-switched B-cell DNA marker comprising a sequence of IGHD at chromosome 14q32.33;
and optionally at least one primer (e.g. at least two primers) and/or a probe for specifically amplifying a DNA regional corrector of the class-switched B-cell DNA marker.

Suitably, the diploid reference DNA marker may be selected from the group consisting of: exon 14 of DNM3, TTC5, TERT, VOPP1.

Suitably, the regional corrector may be selected from the group consisting of: IGHA2, TMEM121, MARK3, BAG5, KLC1, MTA1, CRIP2, PACS2, BRF1, JAG2 and PLD4.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, polynucleotides are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Various aspects of the invention are described in further detail below.

### BRIEF DESCRIPTION OF THE FIGURES

Embodiments of the invention are further described hereinafter with reference to the accompanying figures, in which:

FIG. 1 displays the concept of DNA-based T-cell quantification using ΔB, using the classical or adjusted model described in detail herein. This concept also applies to DNA-based T-cell quantification measurement using ΔD; or DNA-based B-cell quantification using ΔS or ΔH. The classical model can used with samples comprising genetically stable cellular material while the adjusted model is particularly useful for samples comprising genetically unstable cellular material as it adjusts for genomic aberrations affecting the T- or B-cell marker regions that may occur within such samples. Several samples may therefore benefit from the adjusted model described herein as it recognises and corrects for copy number alterations of the T- or B-cell marker loci that are present in admixed not T- or B-cells within the sample.

FIG. 2 shows a proposed clinical workflow that uses the methods of the invention and compares it to the workflow that is currently used in the clinic.

FIG. 3A shows a 2D plot of a duplex digital PCR experiment, in which each dot represents one droplet. On both channels, one assay is measured. On channel 1 positivity for the assay for T-cell marker DELTA_B is measured, on channel 2 positivity for the assay for the diploid reference DNA marker TTC5 is measured. Droplets in the right upper corner are positive for both markers.

FIG. 3B shows the calculated TCF based on the absolute presence of the T-cell marker and the diploid reference DNA marker. After applying a Poisson correction and taking into account the total volume of droplets, the concentrations [DELTA_B] and [TTC5] were calculated. Following the formula

$$TCF = 1 - \frac{[ DELTA\_B ]}{[ TTC 5 ]}$$

the T-cell fraction (TCF) can be determined. In this experiment a healthy PBMC sample was analysed, which presented with a T-cell fraction of 60% as determined with flow cytometry, and a T-cell fraction of 59.7% as determined using the inventors' approach.

FIG. 4 shows a multiplex digital PCR with three references and a T-cell assay. FIG. 4A shows a 2D plot of a multiplex digital droplet (ddPCR) experiment, in which each dot represents one droplet. On both channels, two assays are measured. Channel 1 contains assays for a T-cell marker ΔB and diploid reference DNA marker VOPP1. Channel 2 contains assays for the diploid reference DNA markers TTC5 and TERT.

FIG. 4B shows that the calculated T-cell fractions (TCF) in healthy peripheral blood mononuclear cells (PBMC) sample is consistent using diploid reference DNA markers VOPP1, TTC5 and TERT using the methods described herein. An average T-cell fraction of 59% was calculated, which was in line with the result obtained in the duplex experiment in FIG. 3.

FIG. 5 shows a comparison of the TCF values obtained when the classical and adjusted models are used to calculate TCF in healthy PBMC.

FIG. 6 shows a comparison of the TCF values obtained when the classical and adjusted models are used to calculate TCF in a cancerous uveal melanoma sample. (A and B) Two reference genes on chromosome 5 and 14 (TERT, TTC5) and a regional corrector (BRAF_CNV, 7q) were used to calculate the TCF with B. In this tumour sample gain of 7q has occurred and this affects both ΔB and BRAF_CNV. (C) Using the classical model results in a negative TCF which is impossible. Using the regional corrector (BRAF_CNV) in the adjusted model results in correct estimates of the TCF (0%).

FIG. 7 shows the quantification of VDJ rearranged, class switched and non-switched B-cells by digital PCR.

FIG. 7A shows a diagrammatic representation of the approach used. As genomic VDJ rearrangements and class-switch recombination (CSR) of the IGH@ gene cluster take place, B-cells are genetically different from other cell types. In contrast to non-B-cells, the IGH@ marker ΔH is lost in VDJ rearranged B-cells. In addition, ΔS is specifically lost in switched (memory and plasma) VDJ rearranged B-cells.

FIG. 7B shows a schematic depiction of part of the IGH@ gene cluster. The intergenic sequence, located between gene IGHD7-27 and IGHJ1, represents the ΔH marker. This sequence is biallelically deleted by VDJ rearrangement in the bone marrow during early human B-cell development. The data provided herein indicates that upon B-cell activation, the constant-delta gene (IGHD) is biallelically deleted by CSR in the light zone of germinal centers. The inventors called this marker ΔS.

FIG. 7C shows a workflow of B-cell quantification in a cellular mixture of both B and non-B-cells. After DNA isolation, the cellular context has been lost and all alleles are mixed. Digital PCR is performed to obtain an absolute quantification of B-cell marker ΔH and copy number stable reference gene REF. The absolute loss of ΔH compared to REF reflects the presence of B-cells.

FIG. 8 shows the technical validation standard curves for B-cell quantification using ΔH and ΔS assays on serial DNA dilutions of an enriched B-cell sample and B-lymphocyte cell line. The dilutions of the B cell pool (BCP) and the cell line (L363) are combined to provide extensive coverage.

FIG. 9 To evaluate the mathematical validity of both our classic and adjusted model, an in-silico simulation of the experimental setups was designed.

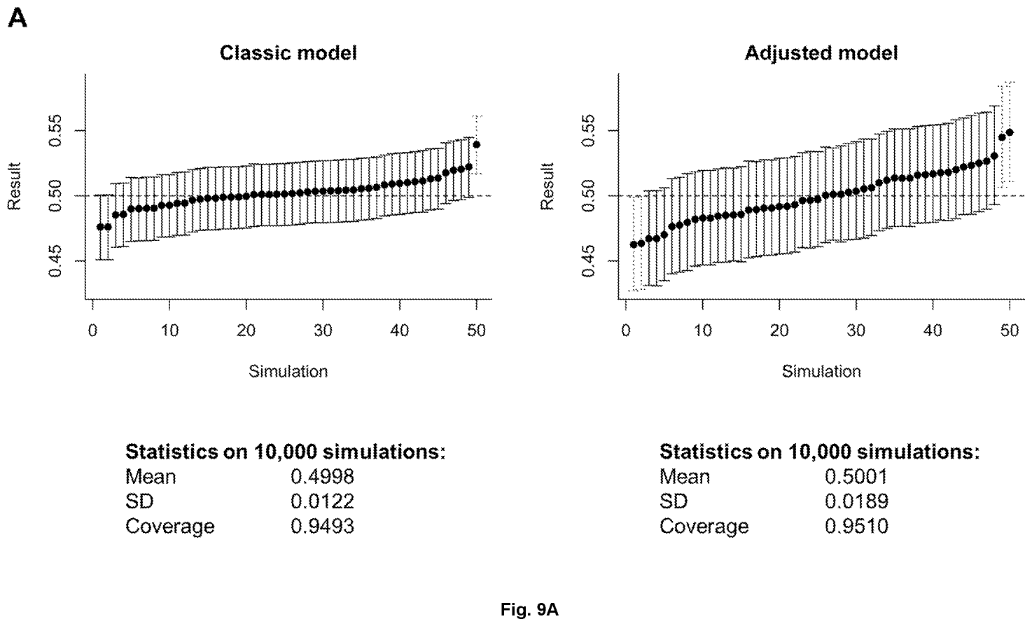

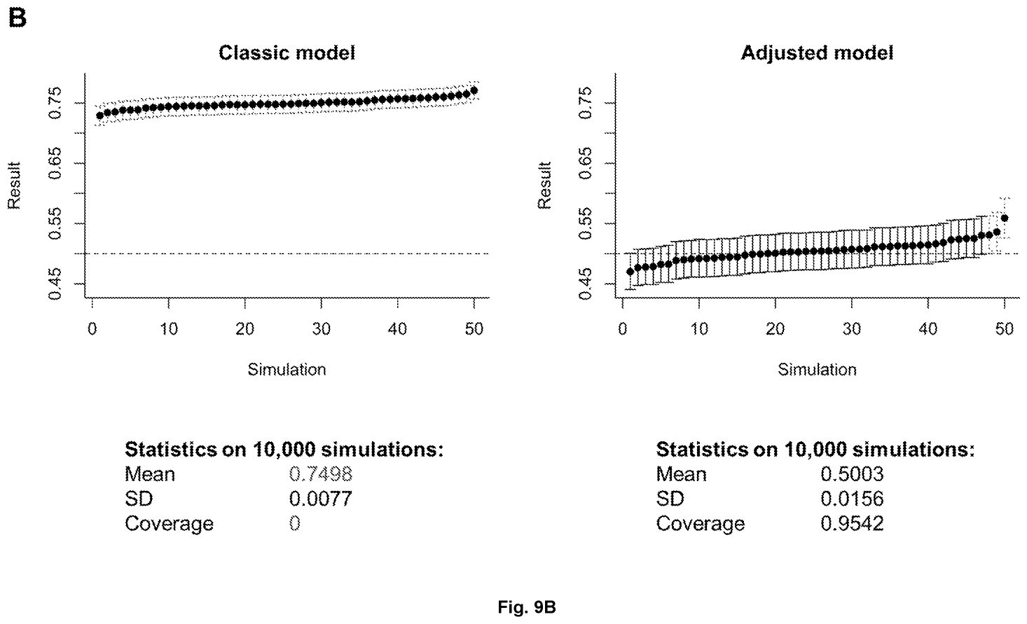

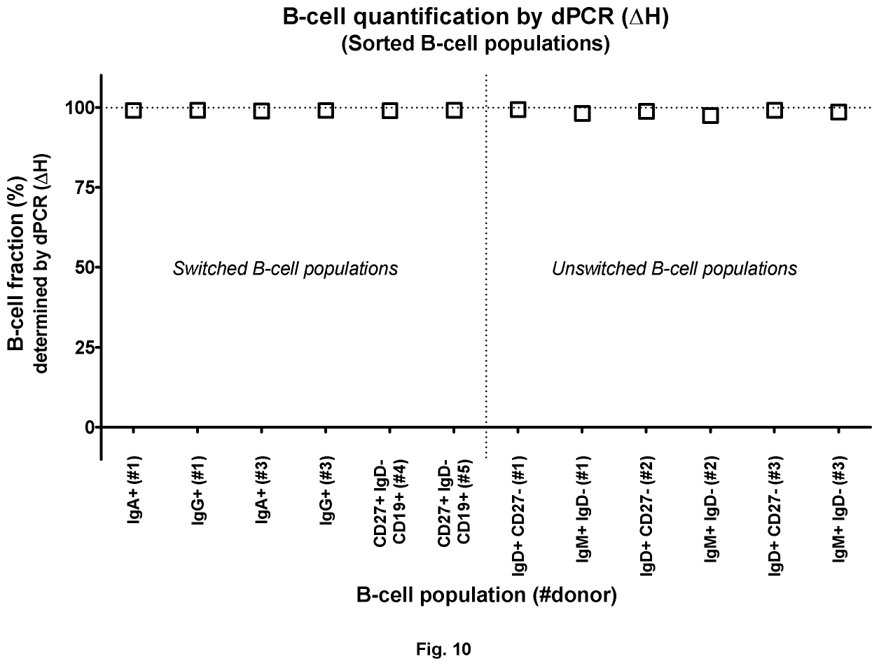

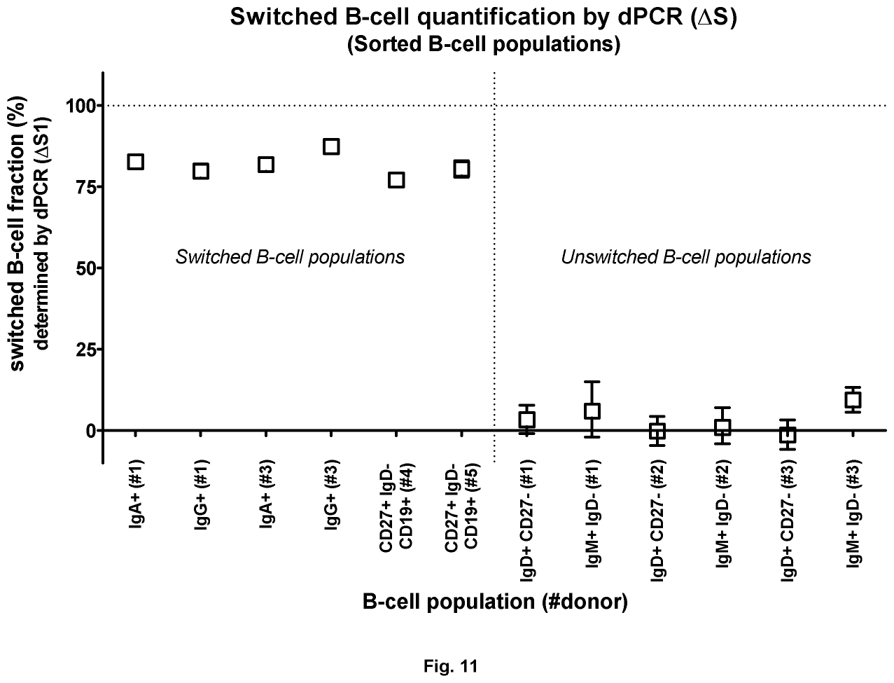

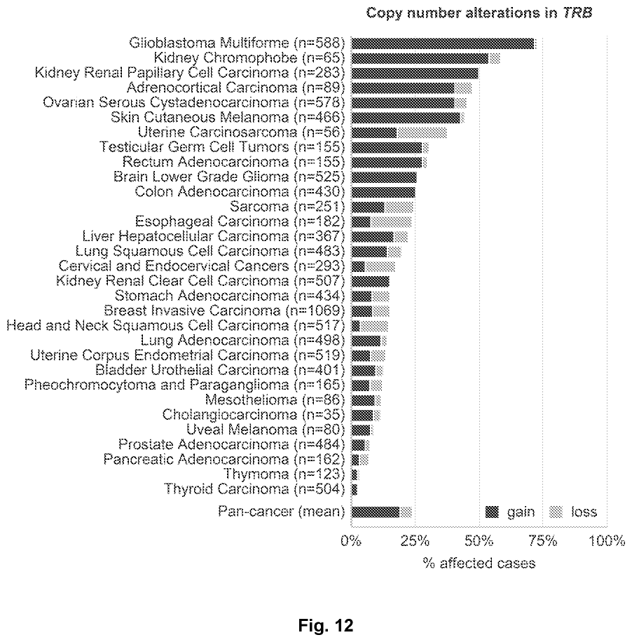

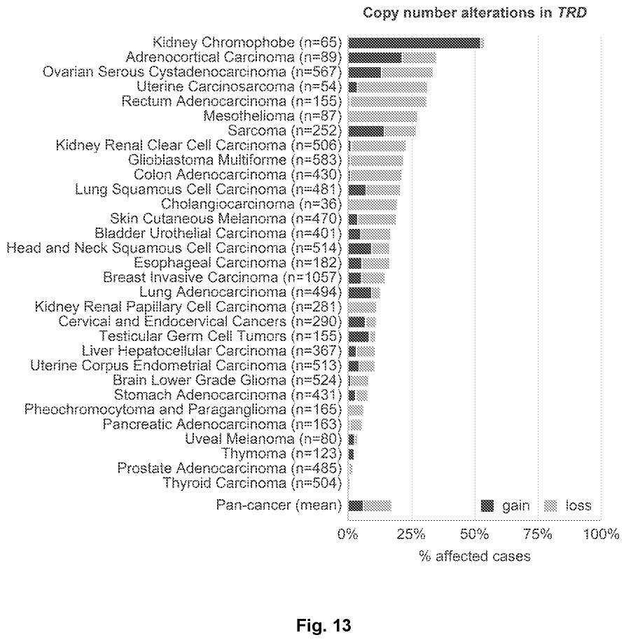

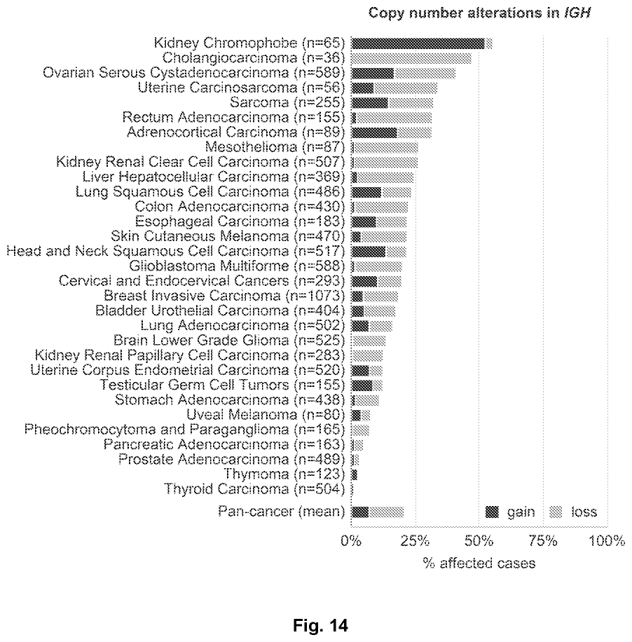

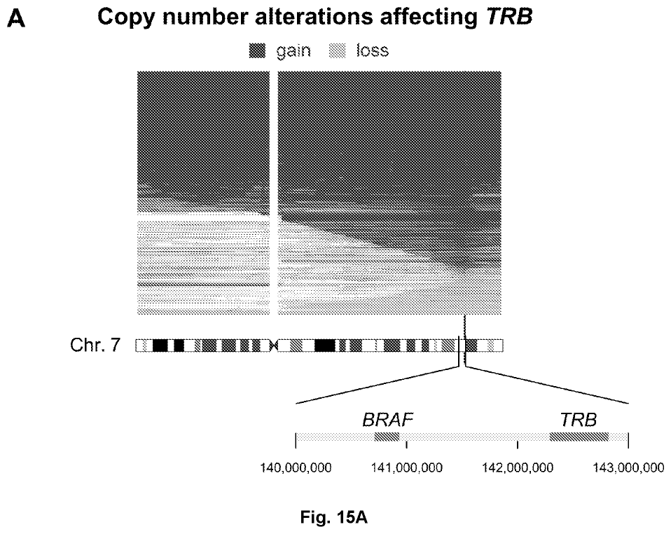

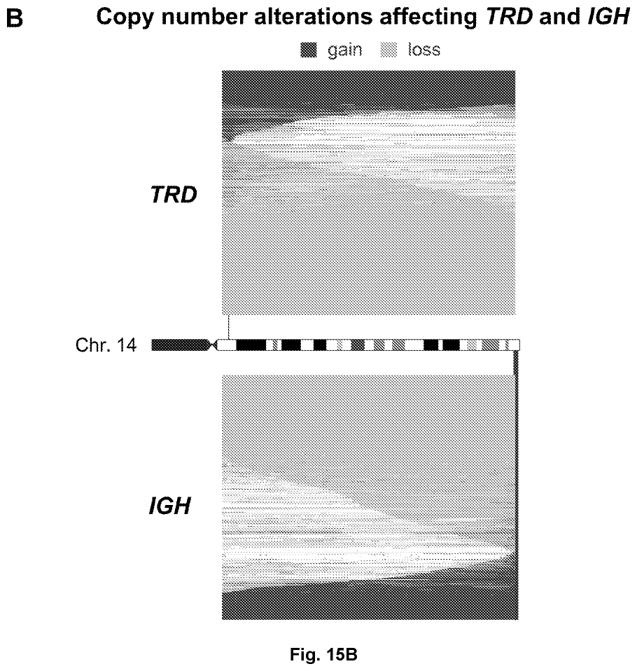

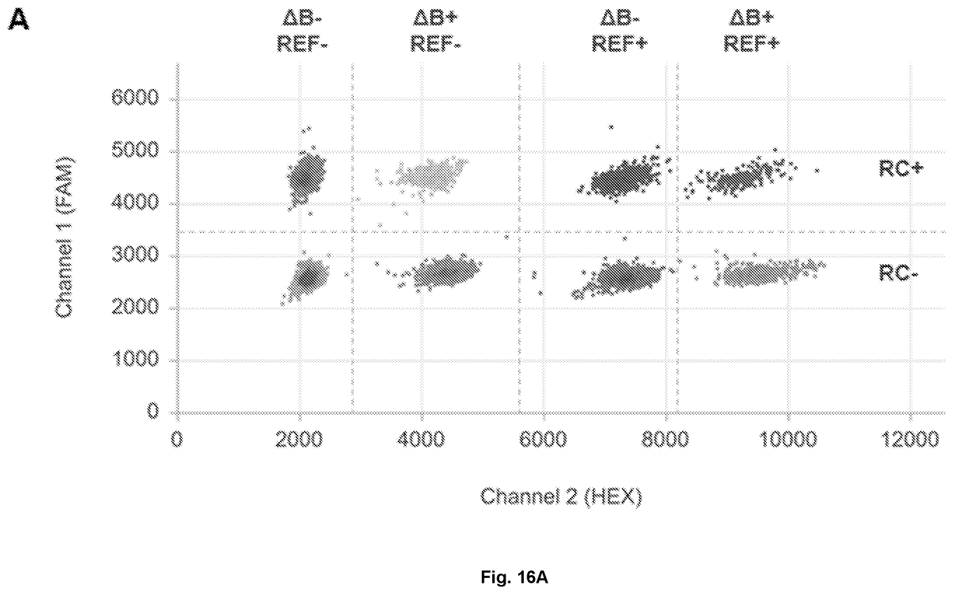

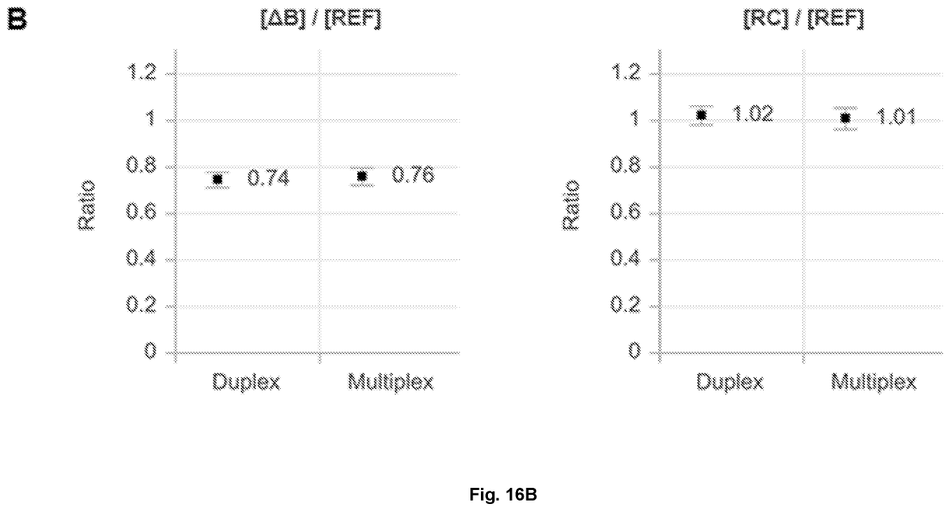

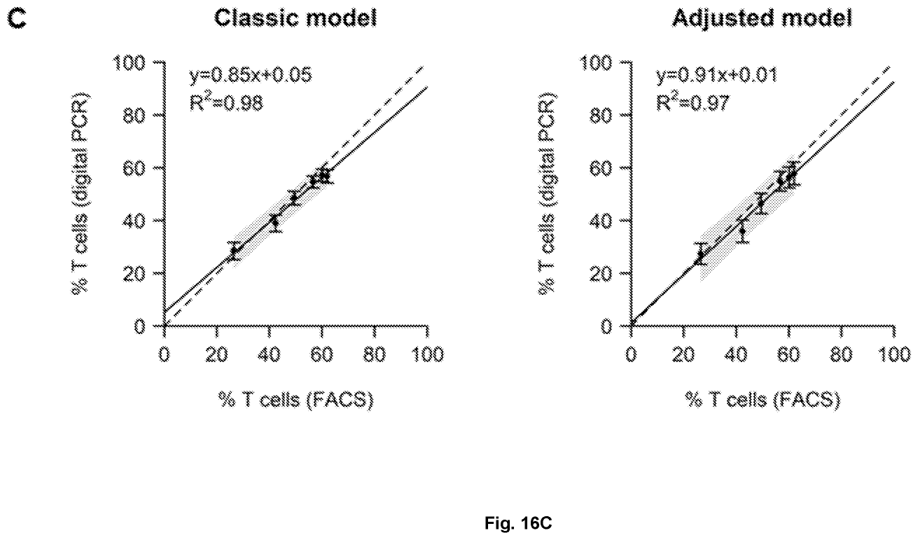

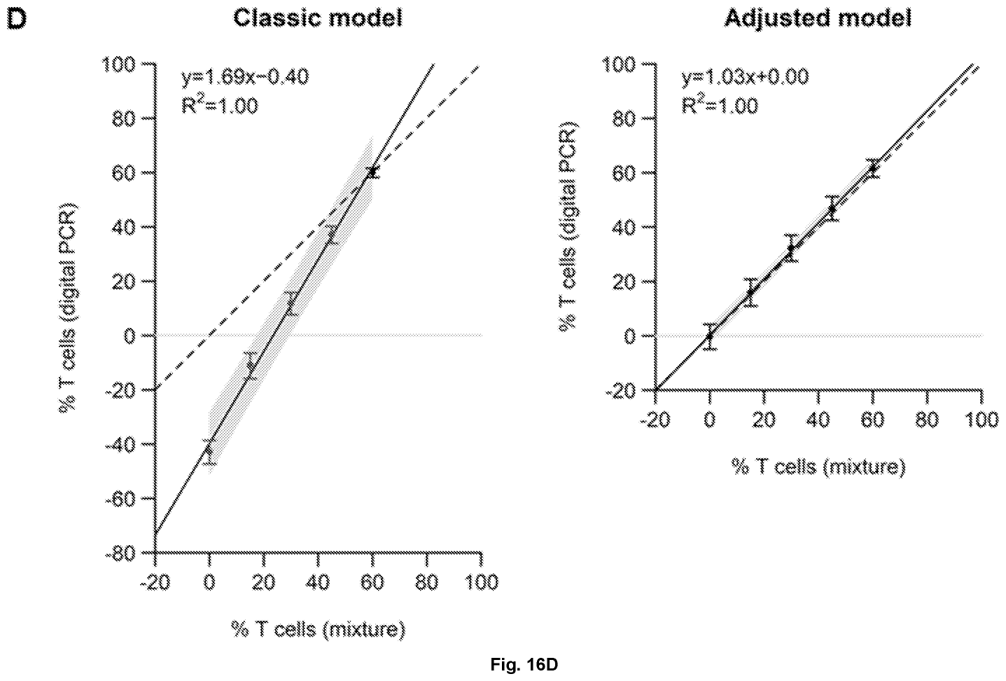

In FIG. 9A, the results of 10,000 in-silico experiments simulating 20 ng copy number stable input DNA and a 50% T-cell fraction are presented. For both the classic and adjusted model, the first 50 calculated T-cell fractions and 95%-confidence intervals are visualized. The overall statistics of all simulations are reported below the plots.

In both models the point estimate has a mean around our true T-cell fraction of 50%. The calculated 95%-confidence intervals generally contain this true T-cell fraction.

Based on all simulations, the 95% CI-coverage (i.e. the percentage calculated 95% confidence intervals that contained the true T-cell fraction) for the classic model is ˜95%. This is close to our intended 95%, demonstrating the mathematical correctness of the classic model in these simulated conditions.

The 95% CI-coverage for the adjusted model is ˜95%. This is close to our intended 95%, demonstrating the mathematical correctness of the adjusted model in these simulated conditions

In FIG. 9B, the results of 10,000 in-silico experiments simulating 20 ng copy number unstable input DNA (complete monosomy at the T-cell marker region in all non-T cells) and a 50% T-cell fraction are presented. For both the classic and adjusted model, the first 50 calculated T-cell fractions and 95%-confidence intervals are visualized. The overall statistics of all simulations are reported below the plots.

In the classic model the loss of one copy of the T-cell marker region in all non-T-cells leads to a total loss of the T-cell marker of 75%, which is indeed observed as the point estimate mean. However, this value does not reflect the correct T-cell fraction. In the adjusted model, the point estimate has a mean around the true T-cell fraction of 50% and the calculated 95% confidence intervals generally contain this true T-cell fraction.

The 95% CI-coverage for the classic model is ˜0%. This means that in none of the experiments the true T-cell fraction was within the calculated interval, indicating the mathematical incorrectness of the classic model in these simulated conditions.

The 95% CI-coverage for the adjusted model is ˜95%. This is close to our intended 95%, demonstrating the mathematical correctness of the adjusted model in these simulated conditions.

FIG. 10 shows digital PCR based quantification of B-cells by measuring loss of marker ΔH in DNA samples from a variety of, flow cytometric sorted, subpopulations of switched and unswitched B cells. Results show that marker ΔH is biallelicly lost in all switched and unswitched B cells.

FIG. 11 shows digital PCR based quantification of switched B-cells by measuring loss of marker ΔS in DNA samples from a variety of, flow cytometric sorted, subpopulations of switched and unswitched B cells. Results show that marker ΔS is present in unswitched B cells, but is lost in approximately 81% of analysed alleles in this cohort of switched B cell samples.

In FIG. 12, copy number alterations in the TRB gene are shown.

In FIG. 13, copy number alterations in the TRD gene are shown.

In FIG. 14, copy number alterations in the IGH gene are shown.

In FIG. 15, the distribution of chromosomal/focal gains (dark grey) and losses (light grey) across chromosome 7 (in cases with an alteration affecting TRB) and chromosome 14 (in cases with an alteration affecting TRD or IGH) is shown. The black vertical line indicates the genomic position of the TRB, TRD or IGH gene.

In FIG. 16, T-cell quantifications are shown according to the classic and adjusted model determined by multiplex digital PCR and compared to flow cytometry. (A) 2D plot of 1×2 multiplex digital PCR analysing RC_{ΔB} on channel 1 (FAM) and ΔB (assay with lowest fluorescence) and REF (assay with highest fluorescence) on channel 2 (HEX), leading to 8 distinct clusters. (B) Comparison of the concentration ratios [ΔB]/[REF] and [RC]/[REF] obtained by separate experiments with conventional duplex configuration and the inventors' combined multiplex setup from (A), showing very similar results. (C) T-cell fractions according to the classic and adjusted model (y-axis) compared to the fractions measured by flow cytometry (x-axis) in 6 healthy, copy number stable PBMC samples. Although accurate quantifications are obtained with both models, the standard deviation is larger when following the adjusted model. (D) T-cell fractions according to the classic and adjusted model (y-axis) compared to the expected fraction (x-axis) in 5 DNA mixtures of a healthy, copy number stable PBMC DNA sample with 60% T-cells and copy number unstable Mel-202 cell line DNA with 0% T-cells. The trisomy of chromosome 7 in Mel-202 results in wrongly calculated T-cell fractions following the classic model, but these are correctly normalised with the adjusted model.

### DETAILED DESCRIPTION

The inventors have developed new DNA-based methods for accurate B- and T-cell quantification. These new methods are based on structural changes at the DNA level that are unique to B- and T-cells. The invention therefore utilises dissimilarities on a genetic level between cell types and/or (sub)populations to accurately quantify different immune cells. The same approach is also able to distinguish between switched and non-switched B-cells which is correlated to B-cell activation.

### Classical Model: Underlying Mathematical Rationale

The inventors have previously identified genetic markers to quantify VDJ rearranged T-cells. These markers are found within the T-cell receptor (TCR) genes, which rearrange during the development of lymphocytes. The concept of using these markers to quantify T-cells has been outlined in an earlier publication and is described briefly below as the “classical model” (Zoutman et al., 2017).

The classical model is based on the finding that T-cells lose specific TCR DNA markers during TCR rearrangement, which occurs as the T-cells develop into mature T-cells. The classical model uses these “lost” TCR DNA markers (also referred to as ΔB and ΔD below) together with genetic markers that are ubiquitous to all cells to determine the T-cell fraction (TCF) in a cell sample. In this context and throughout the text below, “TCF” therefore refers to the fraction of cells in a sample that are VDJ-rearranged T-cells (in other words T-cells that have undergone VDJ rearrangement at the genetic level and thus are in the process of T-cell maturation or have already developed into mature T-cells).

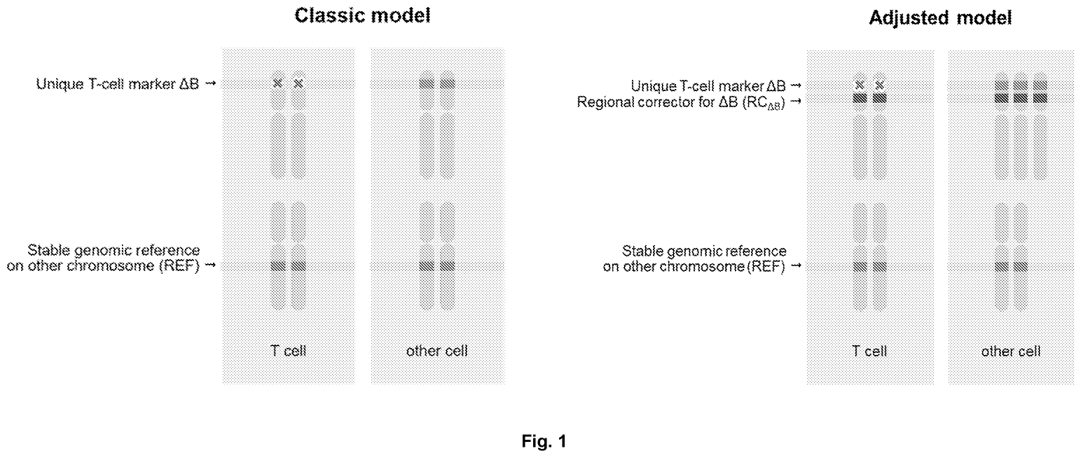

The classical model is based on the fact that all cells are diploid (FIG. 1). In addition, it is based on the fact that during TCR rearrangement, ΔB and ΔD are lost from maturing T-cells. ΔB and ΔD are therefore used in the model as “TCR specific DNA markers” that are lost from the genome of VDJ rearranged T-cells. In essence, these TCR specific DNA markers are therefore negative markers for such VDJ rearranged T-cells (where the absence of the marker indicates the presence of the corresponding T-cell and vice versa). The classical model can advantageously be used in combination with a digital PCR based assay to rapidly and accurately determine the TCF in a sample.

The underlying mathematical rationale of the classical model is as follows, wherein square brackets refer to concentration of the respective target:

ΔB (or ΔD) is present on 0 alleles derived from T-cells, and present on 2 alleles derived from all other cells:

$$\begin{matrix}[ Δ B ] = 0 \cdot TCF + 2 \cdot ( 1 - TCF ) & ( 1 ) \\ = 2 \cdot ( 1 - TCF ) & ( 2 )\end{matrix}$$

REF (also referred to as a “diploid reference DNA marker” herein) is present on 2 alleles derived from T-cells, and present on 2 alleles derived from all other cells:

$$\begin{matrix}[ REF ] = 2 \cdot TCF + 2 \cdot ( 1 - TCF ) & ( 3 ) \\ = 2 & ( 4 )\end{matrix}$$

The ratio

$$\frac{[ Δ B ]}{[ REF ]}$$

can then be rewritten as follows:

$$\begin{matrix}\frac{[ Δ B ]}{[ REF ]} = \frac{2 \cdot ( 1 - TCF )}{2} & ( 5 ) \\ = 1 - TCF & ( 6 )\end{matrix}$$

Which results in the formula to calculate the T-cell fraction:

$$TCF = 1 - \frac{[ Δ B ]}{[ REF ]} \qquad ( 7 )$$

Dube et al. (2008) described how confidence intervals for concentration estimates (i.e. [ΔB] and [REF]) and their ratios can be calculated from digital PCR experiments (Dube et al., 2008). Those approaches are readily applicable on formula 7, by which a TCF and accompanying confidence interval for a given level of significance can be determined.

The above classical model can be used to quantify the fraction of T-cells present in non-malignant cell samples (i.e. samples that do not harbour copy number alterations (CNAs), such as benign cell samples). Validation of the classical model for enumerating T-cells has been performed previously (see (Zoutman et al., 2017)).

The classical model is described above in the context of ΔB. However, the same methodology also applies when using ΔD to quantify T-cells in a sample. As will be described below, the same REF (also referred to as a “diploid reference DNA marker” herein) can be used with either ΔB or ΔD.

More details on ΔB and ΔD are given below.

### Adjusted Model

The inventors have now further developed the classical model to generate an improved method for enumerating T-cells in a sample (referred to herein as the “adjusted model”). Advantageously, the new and improved method can be used for a wide variety of samples with DNA copy number variations, or samples that may be DNA copy number unstable, or samples that originate from cells with copy number instability.

There are several samples that would particularly benefit from T-cell fraction quantification using the adjusted model, where the samples are not malignant in origin. Structural variants of the human genome occur in the natural population and may or may not be inherited. Translocations, both balanced and unbalanced, develop during gametogenesis or during early development and may present as inherited traits or may present as mosaic variation. Gains and deletions in variable sizes occur as well throughout the genome and also may hamper accurate gene copy number quantification.

Historically, variants that were correlated with a disease phenotype and could be recognized with cytogenetics were most studied while other variants remained undefined. The 14q deletion syndrome is such an example of a CNV that may hamper DNA based B cell quantification (Ortigas et al. J Med Genet 34: 515— 517). If the deletions extend into the IGH locus they may underlie immune deficiency that is correlated to frequent respiratory infections.

In current next generation sequencing approaches CNVs are commonly detected but are rarely validated independently. Because of inherent PCR biases of whole exome sequencing and because of CNV software limitations, sensitivity and specificity of CNV calling is low (Fatima Zare et al. BMC Bioinformatics. 2017 May 31; 18(1):286). Most CNVs will therefore remain undefined and this provides a knowledge gap that generates an uncertainty in copy number dependent methods such as the DNA based lymphocyte counting that is presented herein. Advantageously, the adjusted model mitigates this uncertainty by providing the regional corrector approach that corrects for CNVs involving the marker loci of T-cells (or CNVs involving the marker loci of B-cells, see below), such that the copy number variation is corrected for. Because of the uncertainty of CNVs in the population the adjusted model that includes the regional corrector should be the method of choice to measure B and T cells with DNA based methods irrespective of sample origin and/or whether the sample is known to have CNVs or be copy number unstable.

Samples that would benefit from T-cell fraction quantification using the adjusted model include samples of malignant origin. In such samples, genetic stability may be lost and copy number alterations (CNAs) may disturb accurate T-cell quantification. For example, CNAs involving the ΔB marker region or ΔD marker region may lead to a distortion of the classical model. The terms “CNV” and “CNA” are used interchangeably herein.

By including at least two reference loci in the analyses (referred to as (i) a “diploid reference DNA marker” (or REF) and (ii) a regional corrector (RC) herein), the inventors were able to build an advanced digital model, by which genomic aberrations in a sample can be recognized and corrected for. As a result, accurate immune cell quantification can even be achieved in tissues and samples that are genetically unstable. The adjusted model described below therefore utilises one extra target, a regional corrector, to recognize and normalize copy number alterations involving the T-cell marker loci. The adjusted model can advantageously be used in combination with a digital PCR based assay to rapidly and accurately determine the TCF in a sample, including in a sample that is prone to copy number instability, such as a malignant cell sample.

In the mathematical rationale provided for the adjusted model below, the number of extra copies of the ΔB marker region (or ΔD marker region, where appropriate) in in admixed non-T cells is described by “A”.

The mathematical derivation of the adjusted model is summarised as follows:

ΔB is present on 0 alleles derived from T-cells, and present on 2+A alleles derived from other, possibly malignant cells:

$$\begin{matrix}[ Δ B ] = 0 \cdot TCF + ( 2 + A ) \cdot ( 1 - TCF ) & ( 8 ) \\ = ( 2 + A ) \cdot ( 1 - TCF ) & ( 9 )\end{matrix}$$

RC_{ΔB} is present on 2 alleles derived from T-cells and present on 2+A alleles derived from other, possibly malignant cells:

$$[ {RC}_{Δ B} ] = 2 \cdot TCF + ( 2 + A ) \cdot ( 1 - TCF ) \qquad ( 10 )$$

$$\begin{matrix}[ {RC}_{Δ B} ] - [ Δ B ] = 2 \cdot TCF + ( 2 + A ) \cdot ( 1 - TCF ) - ( 2 + A ) \cdot ( 1 - TCF ) & ( 11 ) \\ = 2 \cdot TCF & ( 12 )\end{matrix}$$

REF is present on 2 alleles derived from T-cells, and present on 2 alleles derived from all other cells:

$$\begin{matrix}[ REF ] = 2 \cdot TCF + 2 \cdot ( 1 - TCF ) & ( 13 ) \\ = 2 & ( 14 )\end{matrix}$$

The ratio

$$\frac{[ {RC}_{Δ B} ] - [ Δ B ]}{[ REF ]}$$

can then he rewritten as follows:

$$\begin{matrix}\frac{[ {RC}_{Δ B} ] - [ Δ B ]}{[ REF ]} = \frac{2 \cdot TCF}{2} & ( 15 ) \\ = TCF & ( 16 )\end{matrix}$$

Which results in the formula to calculate the T-cell fraction:

$$TCF = \frac{[ {RC}_{Δ B} ] - [ Δ B ]}{[ REF ]} \qquad ( 17 )$$

As described in Dube et al. (2008), the concentration of a given target, e.g. [ΔB], can be calculated from the observed fraction of digital PCR partitions being positive for this target (p_{ΔB+}) as follows:

$$\begin{matrix}[ Δ B ] = \frac{- log ( 1 - {p}_{Δ B +} )}{V} & ( 18 ) \\ = \frac{- log ( {p}_{Δ B -} )}{V} & ( 19 )\end{matrix}$$

. . . in which V denotes the volume of one droplet.

The numerator of the ratio in (17) can be rewritten as follows:

$$\begin{matrix}[ {RC}_{Δ B} ] - [ Δ B ] = \frac{- log ( {p}_{{RC}_{Δ B} -} )}{V} - \frac{- log ( {p}_{Δ B -} )}{V} & ( 20 ) \\ = \frac{- ( log ( {p}_{{RC}_{Δ B} -} ) - log ( {p}_{Δ B -} )}{V} & ( 21 ) \\ = \frac{- log ( \frac{{p}_{{RC}_{Δ B} -}}{{p}_{Δ B -}} )}{V} & ( 22 )\end{matrix}$$

. . . of which V is a constant value, and

$$- log ( \frac{{p}_{{RC}_{Δ B} -}}{{p}_{Δ B -}} )$$

is a log-ratio of two binomial distributions (see Katz et al, 1978), which is approximately normally distributed with variance:

$$Var = \frac{{p}_{{RC}_{Δ B} -}^{- 1} - 1}{{T}_{{RC}_{Δ B}}} + \frac{{p}_{{RC}_{Δ B} -}^{- 1} - 1}{{T}_{Δ B}} \qquad ( 23 )$$

In which T_{RC}_{_{ΔB}} and T_{ΔB} denote the total number of droplets being analysed in the respective experiments determining [RC_{ΔB}] and [ΔB], which is equal to each other when both targets are measured simultaneously in one experiment:

$$Var = \frac{{p}_{{RC}_{Δ B} -}^{- 1} - 1 + {p}_{Δ B -}^{- 1} - 2}{T} \qquad ( 24 )$$

Wherein, T in formula (24) denotes the total number of droplets or digital PCR partitions. Now, similar to the approaches of Dube et al. (2008), the numerator of (17) can be calculated with accompanying confidence interval, which is used in the construction of the confidence interval of (17), the TCF according to the adjusted model, itself.

When [RC_{ΔB}]=[REF] (i.e. when no CNA is affecting the RC_{ΔB} region), the adjusted model (17) resolves into the classical model (7):

$$\begin{matrix}\frac{[ {RC}_{Δ B} ] - [ Δ B ]}{[ REF ]} = \frac{[ REF ] - [ Δ B ]}{[ REF ]} & ( 25 ) \\ = 1 - \frac{[ Δ B ]}{[ REF ]} & ( 26 )\end{matrix}$$

The inventors demonstrated the mathematical correctness of the classic model and the adjusted model in simulated conditions (FIG. 9).

As for the classical model above, the adjusted model is described above in the context of ΔB. However, the same methodology also applies when using ΔD to quantify T-cells in a sample. As will be described below, the same REF (also referred to as a “diploid reference DNA marker” herein) can be used with either ΔB or ΔD in a sample. However, different regional correctors are to be used with ΔB than ΔD. This is because ΔB is found on chromosome 7 (chr 7q34) and its regional corrector must be located on the same chromosome and in sufficiently close proximity to ΔB. By contrast, ΔD is found on chromosome 14 (chr 14q11.2) and its regional corrector must be located on the same chromosome and in sufficiently close proximity to ΔD. Examples of suitable regional correctors for ΔB and ΔD are found below.

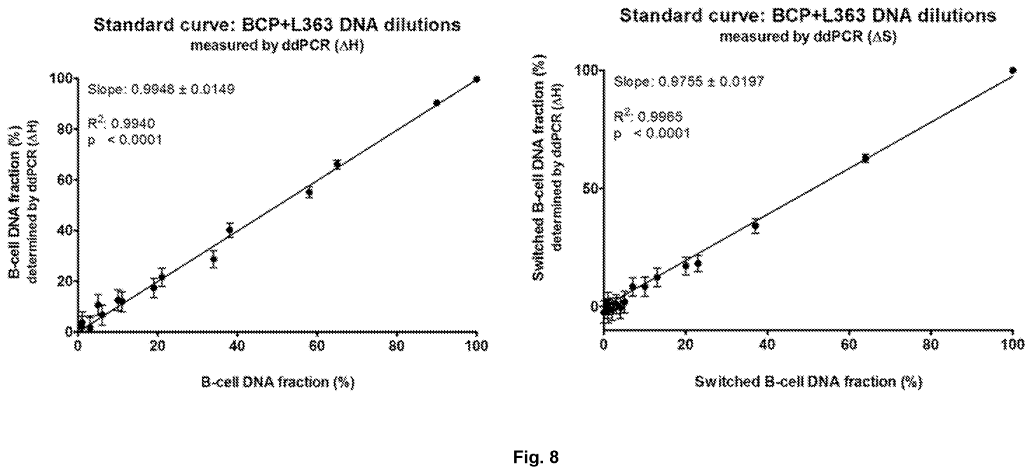

The quantitative nature of the inventors' methods has been validated in in vitro diluted cell types and in a range of control samples that have been analysed in parallel with flow cytometry (which is considered the gold standard for cell quantification). This revealed a high correlation between the methods (see FIG. 8). The methods described herein for T-cells (and B-cells—see below) therefore provide new assays for rapidly and accurately determine the TCF (and B-cell fraction (BCF)—see below) in a sample, including in a sample that may be prone to DNA copy number instability, such as a malignant cell sample.

The classical and adjusted model methods described herein have several advantages over alternative methods known in the art, such as flow cytometry. For example, the methods described herein use genetic DNA markers to quantify immune cells. DNA molecules represent the actual number of immune cells more accurately than transcriptionally and translationally expressed molecular markers (as measured by flow cytometry or immunohistochemistry). Moreover, the amount and integrity of samples required for flow cytometry is much higher than the amount and integrity of the samples needed for the DNA-based quantification methods described herein. For example, flow cytometry requires intact living cells; DNA-based cell quantification on the other hand does not require a cellular context and can be successfully achieved using 5-50 ng degraded DNA, roughly representing 1000-10000 cells.

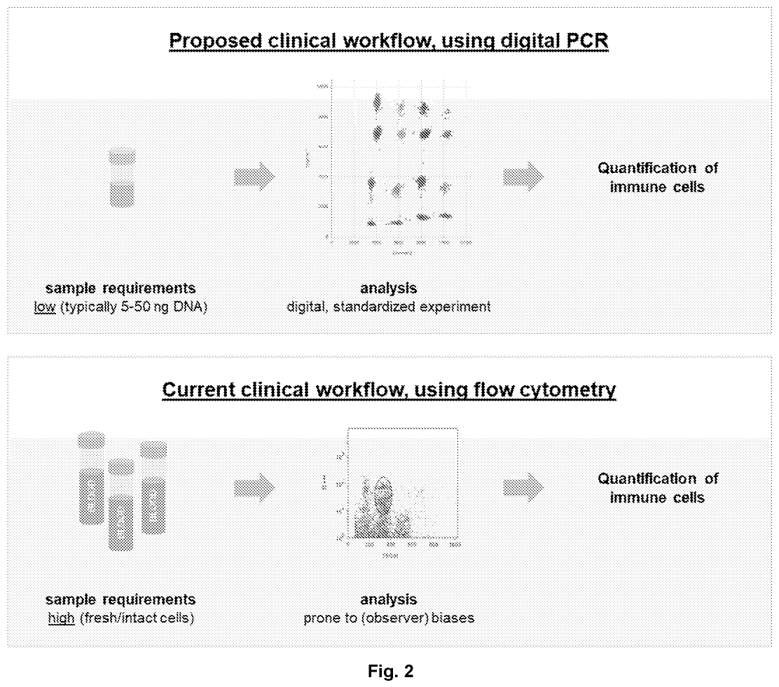

Furthermore, the methods provided herein can be used in combination with a digital PCR based assay to rapidly and accurately determine the TCF in a sample, including in a sample that may be prone to DNA copy number instability such as a malignant cell sample. Direct cell counting without the necessity of a statistical intermediate alleviates standardization and normalization. Digital PCR provides a direct and absolute means of quantification and correspondingly doesn't require standards or calibration curves to perform quantification measurements and calculate accuracy (Vogelstein and Kinzler, 1999). The methods provided herein can therefore advantageously be used for high throughput screening, quantifying immune cells in different sample types that are obtained via minimally invasive methods. A simplified clinical workflow can also be implemented utilising the methods described herein (see FIG. 2).

A further advantage of the methods described herein is that it is possible to calculate corresponding confidence intervals for the TCF (or BCF—see below) value that is generated. This provides the user with a means of quality control which is not available for other methods in the art. This may be crucial in a clinical setting, where decisions on treatment options etc may depend on the accuracy of the immune cell quantification. In such cases, a confidence interval of at least 95%, for example at least 96%, at least 97%, at least 98%, at least 99% etc may be desired.

In addition, for tumour-containing samples (or tumour derived samples), the inventors have found that the adjusted model described herein is particularly advantageous, as it also uses a regional corrector to adjust for possible copy number alterations in the TCR (or BCR) DNA marker region of the test sample. The use of this additional regional corrector is shown herein to increase accuracy in correctly determining the TCF (or BCF) in copy number unstable samples such as malignant cell samples.

### Determining a T-Cell Fraction (TCF) in a Sample

The present invention provides a method for determining the VDJ rearranged human T-cell fraction in a sample. The method is based on the mathematical rationale underlying the adjusted model described above.

The method for determining the VDJ rearranged human T-cell fraction in a sample comprises:
a) quantifying, in the sample, the amount of:
   a diploid reference DNA marker;
   a TCR DNA marker selected from an intergenic region between Dδ2 and Dδ3 on chromosome 14q11.2 or an intergenic region between Dβ1 and Jβ1.1 on chromosome 7q34; and
   a DNA regional corrector of the TCR marker; and
b) determining the VDJ rearranged human T-cell fraction in the sample based on the quantification obtained in step a).

The markers and correctors can be quantified in any order. They can be quantified separately, sequentially or simultaneously, in one or more samples (or sample aliquots). However, multiplexing assays may be preferred as they require only one sample, therefore variation between samples can be excluded.

The term “VDJ rearranged T-cell” refers to T-cells that have already undergone VDJ rearrangement at a genetic level to generate re-arranged adaptive immune receptor genes (i.e. encoding a TCR). Similarly, “VDJ rearranged B-cell” refers to B-cells that have already undergone VDJ rearrangement at a genetic level. “VDJ recombination” is the unique mechanism of genetic recombination that occurs only in developing lymphocytes during the early stages of T-cell and B-cell maturation and is a term widely used in the art. Methods for identifying whether a T-cell or B-cell has undergone VDJ recombination are well known in the art (see for example; (Linnemann et al., 2014; Robins et al., 2013; van Dongen et al., 2003)).

The term “VDJ rearranged T-cells” includes T-cells at different stages of maturation (after VDJ recombination has occurred). For example, it includes T-cells that express a functional T-cell receptor (TCR). It also includes T-cells that have undergone further maturation, for example T-cells that additionally express CD4 and or CD8. The term “VDJ rearranged T-cells” therefore also encompasses mature CD4+ and/or CD8+ T cells. It also encompasses both αβT-cells and γδT-cells. The terms “VDJ-rearranged T-cell” and “mature T-cell” are used interchangeably herein unless the context specifically indicates otherwise.

A specific non-limiting example of VDJ rearranged T-cells that may be detected using the methods described herein are tumour infiltrating lymphocytes (TILs) which may represent helper T cells, memory T cells, effector T cells, and regulatory T cells. The methods described herein may therefore be used to determine the amount of e.g. TIL in a tumour sample or the amount of Tissue Resident Memory (TRM) cells in a normal tissue for example.

In the mathematical formulae above, “TCF” denotes the VDJ rearranged T-cell fraction in a sample. The TCF is calculated by measuring the concentration of specific genetic markers in the sample and applying the mathematical formulae described herein to determine the proportion of cells within the sample that are VDJ rearranged T-cells. The specific genetic markers used in the invention are described in more detail below.

The term “diploid reference DNA marker” refers to any DNA region that is located on a set of chromosomes in a given cell. In other words, it is used to refer to a DNA region which is present in two copies on a pair of chromosomes (i.e. one copy on each of a pair of chromosomes, resulting in two copies in total) in a given cell. The diploid reference DNA marker is therefore located on an autosome and not on an allosome (sex determining chromosome). It is in a region of the genome that is stable and is not prone to copy number alterations. The diploid reference DNA marker used in the methods described herein is a positive genomic marker for all cells in a sample. The diploid reference DNA marker should not be a DNA region of a gene encoding any portion of either a T-cell receptor or a B-cell receptor which may be involved in VDJ recombination as T-cells and/or B-cells mature resulting in the DNA regions of the genes being excised such that they would not be present in all cells within a sample (i.e. they would be missing from adaptive immune cells). The method described herein relies upon the fact that there are two copies of the diploid DNA reference marker in every cell present in a sample. In the mathematical formulae above, “REF” denotes the diploid reference DNA marker of the method. It is also referred to herein as the “genomic reference”. These terms are used interchangeably herein.

The terms “diploid reference DNA marker” and “internal control marker” are used interchangeably herein.

As will be appreciated by a person of average skill in the art, the sample type may influence the diploid reference DNA marker used. For example, a malignant sample may be prone to copy number alterations in a number of different genes and therefore use of the most stable diploid reference DNA marker is preferred. Methods for identifying appropriate diploid DNA reference markers in human cells are well known in the art (Carter et al., 2012). In one example, the diploid reference DNA marker may be exon 14 of DNM3 (chromosome 1 q24.3) or a DNA region thereof. Alternatively, the diploid reference DNA marker can be TTC5 (chromosome 14 q11.2) or a DNA region thereof. As a further alternative, the diploid reference DNA marker could be TERT (chromosome 5 p15.33) or a DNA region thereof. In yet another alternative, the diploid reference DNA marker may be VOPP1 (chromosome 7 p11.2) or a DNA region thereof.). As an alternative the most proximal regional corrector (TRBC2, 7q34) can be used as reference in the classic model, under copy number stable conditions.

A “TCR DNA marker” refers to a DNA region that is modified during VDJ recombination. In the context of the invention, TCR DNA markers are regions of the genome that disappear (i.e. are deleted) during VDJ recombination. The TCR DNA markers used herein are therefore negative genetic markers that are absent in VDJ recombined T-cells (e.g. mature T-cells) but are present in all other cells (including T cells before VDJ recombination, and other cells that are not T cells). The level (or amount) of TCR DNA marker in a sample is therefore inversely proportional to the number of VDJ-rearranged T-cells in the sample. By determining the amount of TCR DNA marker in a sample it is possible to calculate the VDJ-rearranged T-cell fraction in the sample. In the mathematical formulae above, “ΔB” denotes the TCR DNA marker of the invention. However, as explained elsewhere herein, ΔB may be replaced with ΔD. In some methods both ΔB and ΔD may be used in parallel duplex reactions, or simultaneously in a multiplex setup.

The inventors have identified two specific intergenic regions that can act as a TCR DNA marker. Firstly, the intergenic region between Dδ2 and Dδ3 on chromosome 14q11.2 (ΔD). Secondly, the intergenic region between Dβ1 and Jβ1.1 on chromosome 7q34 (ΔB). As will be appreciated by a person of average skill in art, the TCR DNA marker need not comprise the entire intergenic region as described herein. Indeed, the TCR DNA marker can be a DNA region within the intergenic region between Dδ2 and Dδ3 on chromosome 14q11.2 (ΔD). Alternatively, the TCR DNA marker can be a DNA region within the intergenic region between Dβ1 and Jβ1.1 on chromosome 7q34 (ΔB).

The terms “delta B”, “ΔB”, “DELTA_B” and “intergenic region between Dβ1 and Jβ1.1 on chromosome 7q34” are used interchangeably herein. Similarly, the terms “delta D”, “ΔD”, “DELTA_D” and “intergenic region between Dδ2 and Dδ3 on chromosome 14q11.2” are used interchangeably.

As stated previously, in the case of a tumour containing sample (or a tumour derived sample), besides a stable diploid reference DNA marker, an additional regional corrector can be used to adjust for possible copy number alterations in the TCR DNA marker region. The use of a regional corrector is according to the “adjusted model” described above.

The term “DNA regional corrector of the TCR marker”, refers to a DNA marker that is in the local vicinity of the TCR marker used in the method described herein. The identity of the DNA regional corrector therefore depends on which TCR DNA marker is being used. DNA markers are within the “local vicinity” of the TCR marker when they are located on the same chromosomal arm as the TCR marker. The regional corrector is therefore always located on the same chromosomal arm as the TCR marker that is being used in the quantification. The regional corrector is a positive genetic marker (in contrast to the TCR marker). Consequently, whilst the regional corrector is located on the same chromosomal arm (and in close vicinity) of the TCR marker, it must also be sufficiently distal from the TCR marker to avoid being removed from the chromosome during VDJ recombination. For example, the second constant (TRBC2) of the TRB locus is not lost during VDJ rearrangement and qualifies as the ultimate regional corrector for the ΔB driven T-cell quantification. Any other DNA marker on the same chromosomal arm as TRBC2 and ΔB (and located further away from ΔB than TRBC2) may also be suitable regional correctors. In other words, any DNA marker on the same chromosomal arm as TRBC2 and at least the same distance away from ΔB as TRBC2 may be appropriately used as a DNA regional corrector. There is no critical distance at which regional correctors can be found and suitability largely depends on tumour specific characteristics. To assure that a potential regional corrector is stable in the tumour at hand, one can use databases with somatic variations in human cancer to verify the gene stability (e.g. http://atlasgeneticsoncology.org). TRY2P is one of the first candidates but centromeric of this gene are many more candidates. Similarly, for ΔD driven T-cell quantification, SALL2 is not lost during VDJ rearrangement and qualifies as a regional corrector for the ΔD driven T-cell quantification. Any other DNA markers on the same chromosomal arm as SALL2 and ΔD (and located further away from ΔD than SALL2) may also be suitable regional correctors. In the mathematical formulae above, “RC” denotes the DNA regional corrector of the TCR marker of the method as described herein.

The regional corrector of the TCR marker differentiates the classical model from the adjusted model. The purpose of using the regional corrector is to gain an understanding of the copy number status of the TCR DNA marker region in admixed non-T cells in the sample. This allows a correction factor to be applied to account for any copy number alterations of the TCR DNA marker that would otherwise lead to a mis-calculation of the VDJ rearranged human T-cell fraction in a sample using the classical model described herein.

For example, if the TCR DNA marker used is in the intergenic region between Dδ2 and Dδ3 on chromosome 14q11.2 then the regional corrector may also be located on the q-arm of chromosome 14, in close proximity to band 11.2 of chromosome 14 (14q11.2). In this instance, the DNA regional corrector could be METTL3. As a further alternative, the DNA regional corrector could be SALL2. As a final alternative, the DNA regional corrector could be TOX4. Other suitable regional correctors may be identified using methods of the art. The regional corrector may also be a DNA region within CHD8, METTL3, SALL2 or TOX4. However, in several tumours these genes are involved in translocations and other genomic aberrations.

Alternatively telomeric markers may be selected such as DAD1 and OR10G3 that are close enough to function as regional corrector.

As a further example, if the TCR DNA marker used is in the intergenic region between Dβ1 and Jβ1.1 on chromosome 7q34 then the regional corrector is also located on the q-arm of chromosome 7, in close proximity to band 34 of chromosome 7 (7q34). The regional corrector can be proximal of the intergenic region between Dβ1 and Jβ1.1 on chromosome 7q34 but distal of BRAF. Other examples include the DNA regional corrector being TRBC2. As a further alternative, the DNA regional corrector could be BRAF but then it should be verified that BRAF is not amplified in the tumour that is the subject of investigation. As another alternative, the DNA regional corrector could be MOXD2P. As yet another alternative, the DNA regional corrector could be PRSS58. Another alternative is that the DNA regional corrector could be MGAM. Another alternative is that the DNA regional corrector could be TAS2R38. Finally, the DNA regional corrector could be CLEC5A. The regional corrector may also be a DNA region within TRBC2, BRAF, MOXD2P, PRSS58, MGAM, TAS2R38 or CLEC5A. For all these genes it has been shown that they are relatively stable in a range of tumours as can be witnessed in publicly available databases (e.g. http://atlasgeneticsoncology.org) but this should be confirmed for the tumour under investigation.

The methods as described herein can be used to determine the fraction of VDJ rearranged human T-cell fraction in a sample, for example by calculating the difference between the regional corrector and the TCR marker from the intergenic region of Dδ2-Dδ3 on chromosome 14q11.2; as a fraction of the diploid reference DNA marker. Alternatively, the methods described herein can determine the VDJ rearranged T-cell fraction by calculating the difference between the regional corrector of the TCR marker and the TCR marker from the intergenic region of Dδ1-Dδ1.1 on chromosome 7q34; as a fraction of the diploid reference DNA marker.

### Determining the VDJ Rearranged B-Cell Fraction (BCF) in a Sample

The methodology described above for calculating a TCF in a sample can also be used for calculating a B-cell fraction (BCF) in a sample as outlined in more detail below.

The inventors have surprisingly identified that, similar to T-cells, some B-cell DNA markers are biallelically lost in all VDJ rearranged B-cells. Specifically, the inventors have found that a part of the IGH@ gene (referred to as the ΔH region herein) is rearranged biallelically regardless of allelic exclusion and can consequently be used as a genomic B-cell marker. This finding by the inventors means that the classical model can also be used for B-cell quantification, which was not previously thought to be the case. Furthermore, the adjusted model may also be used, and is specifically relevant when quantifying B-cells in a sample that may be prone to DNA copy number instability such as a malignant sample.

A method is therefore provided for determining the VDJ rearranged human B-cell fraction in a sample, the method comprising:
a) quantifying, in the sample, the amount of:
   a diploid reference DNA marker; and
a B-cell DNA marker comprising an intergenic sequence between IGHD7-27 and IGHJ1 at chromosome 14q32.33; and
b) determining the VDJ rearranged human B-cell fraction in the sample based on the quantification obtained in step a).

As stated previously for the T-cell methods, the markers can be quantified in any order. They can be quantified separately, sequentially or simultaneously, in one or more samples (or sample aliquots). However, multiplexing assays may be preferred as they require only one sample, therefore variation between samples can be excluded.

The term “VDJ rearranged B-cell” refers to B-cells that have already undergone VDJ rearrangement at a genetic level. VDJ recombination is described in detail elsewhere herein. The term “VDJ rearranged B-cells” includes B-cells at different stages of maturation (after VDJ recombination has occurred). For example, it includes B-cells that express a functional B-cell receptor (BCR) or antibody. It also includes B-cells that have undergone further maturation, for example B-cells that have undergone class switching during activation. The term “VDJ rearranged B-cells” therefore also encompasses class switched and/or non-switched B cells. The terms “VDJ-rearranged B-cell” and “mature B-cell” are used interchangeably herein unless the context specifically indicates otherwise.

A specific non-limiting example of VDJ rearranged B-cells that may be detected using the methods described herein are plasma cells, regulatory B cells, marginal zone B cells, follicular zone B cells or memory B cells.

In the mathematical formulae for the classical and adjusted models above, “TCF” denotes the VDJ rearranged T-cell fraction in a sample. For the avoidance of doubt, when calculating a BCF using the mathematical formulae described above, the term “TCF” is replaced with “BCF”. In addition, the TCR marker ΔB is replaced with a B-cell DNA marker; namely ΔH. Both of these B-cell DNA markers are described in more detail below.

The BCF is calculated by measuring the concentration of specific genetic markers in the sample and applying the mathematical formulae described herein to determine the proportion of cells within the sample that are VDJ rearranged B-cells. The specific genetic markers used in the invention are described in more detail below.

The term “diploid reference DNA marker” in the context of determining the VDJ rearranged human B-cell fraction in a sample has the same meaning as described above in the context of determining the VDJ rearranged human T-cell fraction in a sample. In the mathematical formulae above, “REF” denotes the diploid reference DNA marker of the method described herein.

A “B-cell DNA marker” as used herein refers to a DNA sequence that is modified during VDJ recombination. More specifically, the B-cell DNA markers disappear (i.e. are deleted) during VDJ recombination. The B-cell DNA markers used herein are therefore negative genetic markers that are absent in VDJ recombined B-cells (e.g. mature B-cells) but are present in all other cells (including B cells before VDJ recombination, and other cells that are not B cells). The level (or amount) of B-cell DNA marker in a sample is therefore inversely proportional to the number of VDJ-rearranged B-cells in the sample. By determining the amount of B-cell DNA marker in a sample it is possible to calculate the VDJ-rearranged B-cell fraction in the sample. In the mathematical formulae above, “ΔB” denotes the TCR DNA marker of the invention. In the context of determination of BCF in the mathematical formulae above, “ΔB” is replaced with “ΔH”.

The inventors have identified one intergenic region that can act as a B-cell DNA marker: the intergenic region between IGHD7-27 and IGHJ1 at chromosome 14q32.33 (ΔH). Therefore, the inventors have surprisingly found the above classical model described above for T-cells can also be used to enumerate B-cells that are present in non-malignant cell samples (i.e. samples that do not harbour copy number alterations (CNAs), such as benign cell samples). As will be appreciated by a person of average skill in art, the B-cell DNA marker need not comprise the entire intergenic region as described herein. Indeed, the B-cell DNA marker can be a DNA region within the intergenic region between IGHD7-27 and IGHJ1 at chromosome 14q32.33 (ΔH). The terms “delta H”, “ΔH”, “DELTA_H” and “intergenic region between IGHD7-27 and IGHJ1 at chromosome 14q32.33” are used interchangeably herein.

The classical model can therefore be used as follows to determine the fraction of VDJ rearranged B-cells in a sample, particularly in a non-malignant cell sample:

$$BCF = 1 - \frac{[ Δ H ]}{[ REF ]} \qquad ( 27 )$$

As outlined above for T-cells, in samples of malignant origin, genetic stability may be lost and CNAs may disturb accurate B-cell quantification. This is because there could be more than two or fewer than copies of the B-cell marker region in the malignant cells. Therefore, CNAs involving the ΔH locus may lead to a distortion of the classical model. In the case of a tumour containing sample (or a tumour derived sample), besides a stable diploid reference DNA marker, an additional regional corrector can therefore be used to adjust for possible copy number alterations in the B-cell DNA marker region. The use of a regional corrector is according to the “adjusted model” described above.

The term “DNA regional corrector of the B-cell DNA marker” refers to a DNA marker that is in the local vicinity of the B-cell DNA marker used in the method described herein. The identity of the DNA regional corrector therefore depends on the B-cell DNA marker used. DNA markers are within the “local vicinity” of the B-cell DNA marker when they are located on the same chromosomal arm as the B-cell DNA marker. The regional corrector is therefore always located on the same chromosomal arm as the B-cell DNA marker that is being used in the quantification. The regional corrector is a positive genetic marker (in contrast to the B-cell DNA marker). Consequently, whilst the regional corrector is located on the same chromosomal arm (and in close vicinity) of the B-cell DNA marker, it must also be sufficiently distal from the B-cell DNA marker to avoid being removed from the chromosome during VDJ recombination. For example, IGHA2 is not lost during VDJ rearrangement or isotype switching (see below) and qualifies as the ultimate regional corrector for the ΔH driven B-cell quantification. Any other DNA markers on the same chromosomal arm as IGHA2 and ΔH (and located further away from ΔH than IGHA2) may also be suitable regional correctors. In other words, any DNA marker on the same chromosomal arm as IGHA2 and at least the same distance away from ΔH as IGHA2 may be appropriately used as a DNA regional corrector. The IGH@ gene is located at the tip of the large arm of chromosome 14. Hence only genes located centromeric of IGH@ gene can suffice as regional corrector. Everything centromeric of IGHA2 could be considered while everything telomeric of IGHA2 must be avoided because these sequences could be involved in VDJ rearrangement for as far as they are part of the IGH@ gene.

In the mathematical formulae above, “RC” denotes the DNA regional corrector of the TCR marker used in the methods described herein. “RC” also refers to the DNA regional corrector of the B-cell DNA marker when the mathematical formulae of the adjusted model is applied in the context of quantifying the BCF in a sample.

Therefore, the BCF can be determined in a sample (e.g. a malignant cell sample) according to the adjusted model by:

$$BCF = \frac{[ {RC}_{Δ H} ] - [ Δ H ]}{[ REF ]} \qquad ( 28 )$$

The regional corrector differentiates the classical model from the adjusted model. The purpose of using the regional corrector is to gain an understanding of the copy number status of the B-cell DNA marker region in the cells in the sample. This allows a correction factor to be applied to account for any copy number alterations of the B-cell DNA marker that would otherwise lead to a mis-calculation of the VDJ rearranged human B-cell fraction in a sample using the classical model described herein.

The regional corrector is also located on the q-arm of chromosome 14. In this instance, the DNA regional corrector could be IGHA2 that is part of the IGH locus but is not lost during isotype switching. Alternatively, the DNA regional corrector could be TMEM121. As a further alternative, the DNA regional corrector could be MARK3. As a further alternative, the DNA regional corrector could be BAG5. As a further alternative, the DNA regional corrector could be KLC1. As another alternative, the DNA regional corrector could be MTA1. As a further alternative, the DNA regional corrector could be CRIP2. As a further alternative, the DNA regional corrector could be PACS2. As a further alternative, the DNA regional corrector could be BRF1. As a further alternative, the DNA regional corrector could be JAG2. As a final alternative, the DNA regional corrector could be PLD4. Other suitable regional correctors may be identified using methods of the art. The regional corrector may also be a DNA region within IGHA2, TMEM121, MARK3, BAG5, KLC1, MTA1, CRIP2, PACS2, BRF1, JAG2 or PLD4.

The regional correctors that have been developed and validated for B-cell counting are the TMEM121 gene and the IGHA2 gene. Whereas TMEM121 is located 50 kb proximal of the IGH locus, the IGHA2 gene is part of the IGH locus but is not lost during isotype switching. Proximal of the IGH locus is a region bordered by MARK3, BAG5 and KLC1 that is involved in translocations and deletions in cancer (Togashi et al., 2012). Regional correctors such as TMEM121 and IGHA2 are distally located of the recombination area and alternative local correctors (MTA1, CRIP2, PACS2, BRF1, JAG2, PLD4) are also located distally of the recombination area.

### Determining the Class-Switched B-Cell Fraction (BCF) in a Sample

The inventors have also developed a B-cell assay that can distinguish between class-switched and non-switched B-cells in a sample using a B-cell marker referred to herein as ΔS. ΔS has been identified as a marker that is rearranged biallelically to differentiate between switched and non-switched B-cells. This finding by the inventors means that the classical model can also be used for quantification of switched and/or non-switched B-cells. Furthermore, the adjusted model may also be used, and is specifically relevant when quantifying switched and/or non-switched B-cells in a sample that may be prone to DNA copy number instability such as a malignant sample.

The classical and adjusted model methodology described above for calculating a VDJ rearranged BCF in a sample can therefore also be used for calculating the fraction of class-switched B-cells in a sample as outlined in more detail below.

A method is therefore provided for determining the class-switched human B-cell fraction in a sample, the method comprising:
a) quantifying, in the sample, the amount of:
   a diploid reference DNA marker; and
   a class-switched B-cell DNA marker comprising a sequence of IGHD at chromosome 14q32.33; and
b) determining the class-switched human B-cell fraction in the sample based on the quantification obtained in step a).

As stated previously, the markers can be quantified in any order. They can be quantified separately, sequentially or simultaneously, in one or more samples (or sample aliquots). However, multiplexing assays may be preferred as they require only one sample, therefore variation between samples can be excluded.

The term “class-switched B-cell” refers to a B cell that has undergone immunoglobulin class switching, also known as isotype switching, isotypic commutation or class-switch recombination (CSR). Class switching is a biological mechanism that changes a B cell's production of immunoglobulin from one type to another, such as from the isotype IgM to the isotype IgG. During this process, the constant-region portion of the immunoglobulin heavy chain is changed, but the variable region of the heavy chain stays the same (the terms “variable” and “constant” refer to changes or lack thereof between immunoglobulins that target different epitopes). Since the variable region does not change, class switching does not affect antigen specificity. Instead, the antibody retains affinity for the same antigens, but can interact with different effector molecules.

In the mathematical formulae for the classical and adjusted models above, “TCF” denotes the VDJ rearranged T-cell fraction in a sample. For the avoidance of doubt, when calculating the class-switched BCF using the mathematical formulae described above, the term “TCF” is replaced with “class switched BCF”. In addition, the TCR marker ΔB is replaced with a class-switched B-cell DNA marker; namely ΔS.

The class-switched BCF is calculated by measuring the concentration of specific genetic markers in the sample and applying the mathematical formulae described herein to determine the proportion of cells within the sample that are VDJ rearranged B-cells. The specific genetic markers used in the invention are described in more detail below.

The term “diploid reference DNA marker” in the context of determining the VDJ rearranged human B-cell fraction in a sample has the same meaning as described above in the context of determining the VDJ rearranged human B-cell fraction in a sample.

A “class switched B-cell DNA marker” as used herein refers to a DNA sequence that is modified during class switching. More specifically, the class switched B-cell DNA markers disappear (i.e. are deleted) during class switching. The class switched B-cell DNA markers used herein are therefore negative genetic markers that are absent in class switched B-cells but are present in all other cells (including B cells before class switching, and other cells that are not B cells). The level (or amount) of the class switching B-cell DNA marker in a sample is therefore inversely proportional to the number of class switched B-cells in the sample. By determining the amount of class switched B-cell DNA marker in a sample it is possible to calculate the class switched B-cell fraction in the sample. In the mathematical formulae above, “ΔB” denotes the TCR DNA marker of the invention. In the context of determination of the class switched BCF in the mathematical formulae above, “ΔB” is replaced with “ΔS”.

The inventors have identified one DNA region that can act as a class switched B-cell DNA marker: a sequence of IGHD at chromosome 14q32.33 (ΔS). Therefore, the inventors have surprisingly found the above classical model described above for T-cells can also be used to enumerate class switched B-cells that are present in non-malignant cell samples (i.e. samples that do not harbour copy number alterations (CNAs), such as benign cell samples). As will be appreciated by a person of average skill in art, the class switched B-cell DNA marker need not comprise the entire sequence of IGHD. Indeed, the class switched B-cell DNA marker can be a DNA region within IGHD (ΔS). The terms “dS”, “delta S”, “ΔS”, “DELTA_S” and “a sequence of IGHD at chromosome 14q32.33” are used interchangeably herein.

The classical model can therefore be used as follows to determine the fraction of class switched B-cells in a sample, particularly in a non-malignant cell sample:

$$class switched BCF = 1 - \frac{[ Δ S ]}{[ REF ]} \qquad ( 29 )$$

As described in the examples section below, the inventors have identified that ΔS is not ubiquitously lost in either a pure mono- or biallelic fashion in benign, uncultured and polyclonal B-cell samples. For example, the inventors found that ΔS was consistently lost in on average 81% (i.e. a fraction of 0.81) of the measured alleles in DNA specimens from switched B cells (see FIG. 11). They have therefore also identified a means for further optimising the classical model when quantifying the B-cell fraction, by the introduction of an allelic factor which can be used to correct for biological imbalances. As will be clear to a person of skill in the art, the allelic factor is the fraction of ΔS/alleles that have been lost in an average switched B-cell (and thus is a correction factor that can be used when quantifying switched B-cells). The allelic factor will be calculated for each class switched B-cell DNA marker separately (in a way that is shown herein for ΔS).

By adding an allelic-factor (AF) to the formula for quantification of switched B cells, the inventors are able to enumerate these cells more accurately in DNA samples by using dPCR.

The formula to quantify switched B-cells is:

[switched B cells]=(1−[dS]/[Ref])

By adding an allelic-factor (AF), the formula turns into:

[switched B cells]=(1−[dS]/[Ref])/AF

To apply this formula on the cohort of samples as described above, the formula will be:

[switched B cells]=(1−[dS]/[Ref])/0.81

More details on how to quantify an appropriate allelic factor for use in with the classical model is provided below. Square brackets refer to concentration of the respective target, SF refers to the switched B-cell fraction. AF denotes the allelic factor, the fraction of alleles that have lost the ΔS marker in switched B-cells.

The classical model can be used to determine the switched B-cell fraction in a sample (without allelic factor) based on the following principles:

ΔS is present on 0 alleles derived from switched B cells, and present on 2 alleles derived from all other cells:

$$\begin{matrix}[ Δ S ] = 0 \cdot SF + 2 \cdot ( 1 - SF ) \\ = 2 \cdot ( 1 - SF )\end{matrix}$$

REF (also referred to as a “diploid reference DNA marker” herein) is present on 2 alleles derived from switched B cells, and present on 2 alleles derived from all other cells:

$$\begin{matrix}[ REF ] = 2 \cdot SF + 2 \cdot ( 1 - SF ) \\ = 2\end{matrix}$$

The ratio

$$\frac{[ Δ S ]}{[ REF ]}$$

can then be rewritten as follows:

$$\begin{matrix}\frac{[ Δ S ]}{[ REF ]} = \frac{2 \cdot ( 1 - SF )}{2} \\ = 1 - SF\end{matrix}$$

Which results in the formula to calculate the switched B-cell fraction:

$$SF = 1 - \frac{[ Δ S ]}{[ REF ]}$$

Alternatively, the classical model can be used to determine the switched B-cell fraction in a sample with an allelic factor, based on the following principles:

ΔS is present on 2·(1−AF) alleles derived from switched B cells. For example, with AF set to 0.81, 1−0.81=19% of the ΔS alleles are still present in switched B cells, which is on average 19%·2=0.38 allele per switched B-cell. ΔS is present on 2 alleles derived from all other cells:

[ΔS]=2·(1−AF)·SF+2·(1−SF)

REF (also referred to as a “diploid reference DNA marker” herein) is present on 2 alleles derived from switched B cells, and present on 2 alleles derived from all other cells:

$$\begin{matrix}[ REF ] = 2 \cdot SF + 2 \cdot ( 1 - SF ) \\ = 2\end{matrix}$$

The ratio

$$\frac{[ Δ S ]}{[ REF ]}$$

can then be rewritten as follows:

$$\begin{matrix}\frac{[ Δ S ]}{[ REF ]} = \frac{2 \cdot ( 1 - AF ) \cdot SF + 2 \cdot ( 1 - SF )}{2} \\ = ( 1 - AF ) \cdot SF + ( 1 - SF ) \\ = SF - AF \cdot SF + 1 - SF \\ = 1 - AF \cdot SF\end{matrix}$$

Which results in the formula to calculate the switched B-cell fraction:

$$AF \cdot SF = 1 - \frac{[ Δ S ]}{[ REF ]} \\ SF = \frac{1 - \frac{[ Δ S ]}{[ REF ]}}{AF}$$

As outlined above for T-cells, in samples that are prone to DNA copy number instability, such as samples of malignant origin, genetic stability may be lost and CNAs may disturb accurate B-cell quantification. This is because there could be more than two or fewer than two copies of the B-cell marker region in the malignant cell. Therefore, CNAs involving the ΔS locus may lead to a distortion of the classical model. In the case of a tumour containing sample (or a tumour derived sample), besides a stable diploid reference DNA marker, an additional regional corrector can therefore be used to adjust for possible copy number alterations in the class switched B-cell DNA marker region. The use of a regional corrector applies the “adjusted model” described above.

The term “DNA regional corrector of the class-switched B-cell DNA marker” refers to a DNA marker that is in the local vicinity of the class switched B-cell DNA marker used in the method described herein. The explanation provided above regarding DNA regional correctors of the B-cell DNA markers applies equally here, as the regional corrector for class-switching can be the same as the regional corrector for the B-cell DNA marker. Therefore, the class switched BCF can be determined in a sample (e.g. a malignant cell sample) according to the adjusted model by:

$$class switched BCF = 1 - \frac{[ {RC}_{Δ S} ] - [ Δ S ]}{[ REF ]}$$

An allelic factor (AF) may also be added to the adjusted model formula for quantification of switched B-cells, in order to enumerate these cells more accurately in DNA samples using dPCR. More details on how to quantify an appropriate allelic factor for use in with the adjusted model is provided below. Square brackets refer to concentration of the respective target, SF refers to the switched B-cell fraction. AF denotes the allelic factor, the fraction of alleles that have lost the ΔS marker in switched B-cells.

The adjusted model may be used to calculate the switched B-cell fraction in a sample using an allelic factor, based on the following principles:

In the adjusted model three DNA markers are quantified: the switched B-cell marker ΔS, regional corrector RC_{ΔS} and a copy-number stable, independent genomic reference REF.

ΔS is present on 2·(1−AF) alleles derived from switched B-cells, and present on 2+A alleles derived from other, possibly malignant cells:

[ΔS]=2·(1−AF)·SF+(2+A)·(1−SF)

RC_{ΔS} is present on 2 alleles derived from switched B-cells and present on 2+A alleles derived from other, possibly malignant cells:

[RC_{ΔS}]=2·SF+(2+A)·(1−SF)

The ratio [RC_{ΔS}]−[ΔS] can then be rewritten as follows:

$$\begin{matrix}[ {RC}_{Δ S} ] - [ Δ S ] = ( 2 \cdot SF + ( 2 + A ) \cdot ( 1 - SF ) ) - ( 2 \cdot ( 1 - AF ) \cdot SF + ( 2 + A ) \cdot ( 1 - SF ) ) \\ = 2 \cdot SAF + ( 2 + A ) \cdot ( 1 - SF ) - 2 \cdot ( 1 - AF ) \cdot SF - ( 2 + A ) \cdot ( 1 - SF ) \\ = 2 \cdot SF - 2 \cdot ( 1 - AF ) \cdot SF\end{matrix}$$

REF is present on 2 alleles derived from T-cells, and present on 2 alleles derived from all other cells:

$$\begin{matrix}[ REF ] = 2 \cdot SF + 2 \cdot ( 1 - SF ) \\ = 2\end{matrix}$$

The ratio

$$\begin{matrix}\frac{[ {RC}_{Δ S} ] - [ Δ S ]}{[ REF ]} = \frac{2 \cdot SF - 2 \cdot ( 1 - AF ) \cdot SF}{2} \\ = SF - ( 1 - AF ) \cdot SF \\ = SF - AF + AF \cdot SF \\ = AF \cdot SF\end{matrix}$$

can then be rewritten as follows:

$$\frac{[ {RC}_{Δ S} ] - [ Δ S ]}{[ REF ]}$$

Which results in the formula to calculate the switched B-cell fraction according to the adjusted model:

$$AF \cdot SF = \frac{[ {RC}_{Δ S} ] - [ Δ S ]}{[ REF ]} \\ SF = \frac{\frac{[ {RC}_{Δ S} ] - [ Δ S ]}{[ REF ]}}{AF}$$

The regional corrector for class switching is therefore also located on the q-arm of chromosome 14. In this instance, the DNA regional corrector could be IGHA2 that is part of the IGH locus but is not lost during isotype switching. Alternatively, the DNA regional corrector could be TMEM121. As a further alternative, the DNA regional corrector could be MARK3. As a further alternative, the DNA regional corrector could be BAG5. As a further alternative, the DNA regional corrector could be KLC1. As another alternative, the DNA regional corrector could be MTA1. As a further alternative, the DNA regional corrector could be CRIP2. As a further alternative, the DNA regional corrector could be PACS2. As a further alternative, the DNA regional corrector could be BRF1. As a further alternative, the DNA regional corrector could be JAG2. As a final alternative, the DNA regional corrector could be PLD4. Other suitable regional correctors may be identified using methods of the art. The regional corrector may also be a DNA region within IGHA2, TMEM121, MARK3, BAG5, KLC1, MTA1, CRIP2, PACS2, BRF1, JAG2 or PLD4.

### Combining ΔS and ΔH

It may be beneficial to use both ΔS and ΔH markers simultaneously, for example to quantify the fraction of non-switched VDJ rearranged B-cells in a sample. The methodology outlined above would still apply. For example, ΔH may be used to quantify the total fraction of VDJ rearranged B cells in the sample and ΔS may then be used to determine what proportion of these B-cells are switched and non-switched. The invention therefore provides for such combination methods (using either the classical model or the adjusted model). Multiplex digital PCR is particularly useful for combining these assays.

### Samples and Diseases

The methods described herein determine the VDJ rearranged B-cell or T-cell fraction in a sample (or alternatively the switched and/or non-switched B cell fraction in a sample).

As used herein “sample” refers to any specimen from a biological source. In some instances, the sample could be obtained from a subject.

The terms “individual”, “subject,” “host” and “patient” are used interchangeably herein and refer to any subject for whom diagnosis, treatment or therapy is desired. For the purposes of the present disclosure, the subject may be a primate, preferably a human, or another mammal, such as a dog, cat, horse, pig, goat, or bovine, and the like. All higher vertebrates that possess VDJ based adaptive immunity are eligible for DNA-based B- and T-cell counting. The subject, from which the sample may be obtained, can be a human or non-human animal, or a transgenic or cloned or tissue-engineered (including through the use of stem cells) organism.

The subject may be a human. The subject, from which the sample is obtained, may be known to have, or may be suspected of having or being at risk for having, a lymphoid hematopoietic cancer or other malignant condition, or an autoimmune disease, or an inflammatory condition. Alternatively, the subject may be known to be free of a risk or presence of such disease. A subject can be a human subject such as a patient that has been diagnosed as having or being at risk for developing or acquiring cancer according to clinical diagnostic criteria, such as those of the U.S. National Cancer Institute (Bethesda, MD, USA) or as described in DeVita, Hellman, and Rosenberg's Cancer: Principles and Practice of Oncology (2008, Lippincott, Williams and Wilkins, Philadelphia/Ovid, New York); Pizzo and Poplack, Principles and Practice of Pediatric Oncology (Fourth edition, 2001, Lippincott, Williams and Wilkins, Philadelphia/Ovid, New York); Vogelstein and Kinzler, The Genetic Basis of Human Cancer (Second edition, 2002, McGraw Hill Professional, New York); Dancey et al. (2009 Semin. Oncol. 36 Suppl.3:S46). Therefore, the human subject can be known to be free of a risk for having, developing or acquiring cancer by such criteria.

The sample could be a tissue sample. The sample could be a body fluid sample. The sample may comprise cells that may be prone to DNA copy number instability such as a malignant cells. As used herein the term “malignant” refers to cells with genomic instability or genomic aberrations. For example, the sample may comprise malignant cells that contain DNA with copy number alterations (i.e. greater than or fewer than two copies of the genome or a portion therefore in a cell) of chromosome 14q. Alternatively or additionally, the sample may comprise cells that contain DNA copy number alterations (i.e. greater than or fewer than two copies) of chromosome 7q.

The sample may comprise all or a portion of a tumour that contains adaptive immune cells and cells that are not adaptive immune cells (including tumour cells). The sample, may take the form of a variety of tissue and biological fluid samples including bone marrow, thymus, lymph glands, lymph nodes, peripheral tissues and blood, but peripheral blood is most easily accessed. Any peripheral tissue can be sampled for the presence of B- and T-cells and is therefore contemplated for use in the methods described herein. Tissues and biological fluids from which adaptive immune cells may be obtained include, but are not limited to, skin, epithelial tissues, colon, spleen, a mucosal secretion, oral mucosa, intestinal mucosa, vaginal mucosa or a vaginal secretion, cervical tissue, ganglia, saliva, eye, eye fluids, cerebrospinal fluid (CSF), bone marrow, cord blood, serum, serosal fluid, plasma, lymph, urine, ascites fluid, pleural fluid, pericardial fluid, peritoneal fluid, abdominal fluid, culture medium, conditioned culture medium or lavage fluid. Peripheral blood samples may be obtained by phlebotomy from subjects. Peripheral blood mononuclear cells (PBMC) are isolated by techniques known to those of skill in the art, i.e. by Ficoll-Hypaque<®> density gradient separation. In some instances, whole PBMCs can be used as the sample. The sample may also comprise all or a portion of a somatic tissue that contains adaptive immune cells and cells that are not adaptive immune cells, such as cells of a solid tissue.

The sample may be processed before the determination of the VDJ rearranged B-cell or T-cell fraction (or switched and/or non-switched B-cell fraction). For example, DNA may be extracted from a mixed population of cells from a sample, such as any neoplastic tissue sample or a sample of somatic tissue that is the target of an autoimmune reaction, blood sample, or cerebrospinal fluid, using standard methods or commercially available kits known in the art. Illustrative samples for use in the present methods include any type of solid tumour, in particular, from colorectal, eye, skin, hepatocellular, gallbladder, pancreatic, esophageal, lung, breast, prostate, head and neck, renal cell carcinoma, ovarian, endometrial, cervical, bladder and urothelial cancers. Any solid tumour in which tumour-infiltrating lymphocytes are to be assessed is contemplated for use in the present methods. Somatic tissues that are the target of an autoimmune reaction that are contemplated for analysis using the methods herein include, but are not limited to, joint tissues, skin, intestinal tissue, all layers of the uvea, heart, brain, lungs, blood vessels, liver, kidney, nerve tissue, muscle, spinal cord, pancreas, adrenal gland, tendon, mucus membrane, lymph node, thyroid, endometrium, connective tissue, and bone marrow. In some instances DNA may be extracted from a transplanted organ, such as a transplanted liver, lung, kidney, heart, spleen, pancreas, skin, intestine, and thymus.

The methods described herein can be used as a multiplex assay. The term “multiplex” is used herein to refer to any assay in which a plurality of parameters are determined in a single sample (i.e. the diploid reference DNA marker and/or the TCR DNA marker and/or the DNA regional corrector and/or or the B-cell DNA marker etc) are determined in a combined experiment from one sample.

Any suitable method can be used to determine the concentration of specified genetic markers (diploid reference DNA marker, TCR DNA marker, DNA regional corrector, and/or B-cell DNA marker etc) in a test sample. Digital PCR may preferably be used in the context of the invention as it allows for absolute quantification of the specified genetic markers in the sample.

In digital PCR, the PCR reaction for a single sample is performed in a multitude of thousands droplets (partitions) by limiting dilution (also referred to herein as “assay samples”), such that each droplet either amplifies (i.e. generation of an amplification product provides evidence of the presence of at least one template molecule in the droplet) or fails to amplify (evidence that the template was not present in a given droplet). Hence, the individual readout signals are qualitative or “digital” in nature. By simply counting the number of positive drops, it is possible directly to count the number of target alleles that are present in an input sample. Digital PCR methods typically use an endpoint readout, rather than a conventional quantitative PCR signal that is measured after each cycle in the thermal cycling reaction (see i.e. (Pekin et al., 2011; Pohl and Shih Ie, 2004; Tewhey et al., 2009; Vogelstein and Kinzler, 1999; Zhong et al., 2011). Compared with traditional PCR, digital PCR has the following advantages: (1) there is no need to rely on references or standards, (2) desired precision may be achieved by increasing the total number of PCR replicates, (3) it is highly tolerant to inhibitors, (4) it is capable of analysing complex mixtures, and (5) it provides a linear response to the number of copies present in a sample to allow for small change in the copy number to be detected.

Digital PCR may therefore be used to quantify the VDJ rearranged human T-cell and/or B-cell fraction in a sample that comprises a mixture of cells (i.e. both adaptive immune cells and cells that are not adaptive immune cells). The method may comprise first distributing test sample template DNA extracted from the sample to form a set of sample partitions followed by amplifying the test sample template DNA in the set of assay samples in a multiplex dPCR. Multiplex dPCR comprises measuring a plurality of markers simultaneously, typically using one DNA sample. Multiplex assays are advantageous as they reduce the need for normalisation across multiple samples. Further details of methodology that can be used for multiplex dPCR is found in, for example, (Whale et al., 2016). Experimental details of the dPCR methods used by the inventors are also provided below.

Any systems known in the art for performing digital PCR methodology may be used in the methods provided herein, for example, the ABI QuantStudio™ 12K Flex System (Life Technologies, Carlsbad, CA), the QX100 or QX200<IM> Droplet Digital™ PCR system (BioRad, Hercules, CA), the QuantaLife™ digital PCR system (BioRad, Hercules, CA), or the RainDance™ microdroplet digital PCR system (RainDance Technologies, Lexington, MA).

The methods described herein can be used for monitoring disease progression. Alternatively, the methods described herein can be used for determining the effect of a medicament used in the treatment of a disease (i.e. identify if a subject is responsive or sensitive to the treatment provided). A further alternative use for the methods described herein are for determining disease prognosis. Finally the methods described herein could be used for diagnosing a disease.

As used herein, a subject is “responsive” or “sensitive” to treatment if they respond therapeutically such that the disease is alleviated or abrogates. This means that the life expectancy of an individual affected with the disease will be increased, or that one or more of the symptoms of the disease will be reduced or ameliorated. For example, the term encompasses a reduction in cancerous cell growth or tumour volume. Whether a mammal responds therapeutically can be measured by many methods well known in the art, such as PET imaging.

The terms “treating” and “therapy” are used interchangeably herein to refer to reducing, ameliorating or eliminating one or more signs, symptoms, or effects of a disease or condition. The terms “therapy” and “treating” are used in the broadest sense and is construed to encompass any medical intervention that is intended to prevent a medical condition from occurring, or to reduce the medical condition to manifest, or to seek to cure the root cause of the disease, or any variations of the foregoing. The terms “preventing” or “prevention” is used here to refer to stopping or reducing the likelihood of the development of symptoms associated with the disease.

As used herein, the “administration” or “administering” of a pharmaceutical composition described herein to a subject includes any route of introducing or delivering to a subject which allows for the composition to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another. The composition can be administered as a therapeutically effective amount. As used herein, the phrase “therapeutically effective amount” means a dose or plasma concentration in a subject that provides the specific pharmacological effect for which the described compositions are administered, i.e. to treat a disease of interest in a target subject. The therapeutically effective amount may vary based on the route of administration and dosage form, the age and weight of the subject, and/or the disease or condition being treated.

There are a wide range of diseases in which immune cell monitoring is worthwhile. Indeed, any disease that either triggers or dampens an immune cell response can be monitored by the methods described herein. Examples include, but are not limited to, infectious diseases, autoimmune diseases, transplantation medicine and cancer.

Autoimmune diseases include, but are not limited to, arthritis (including rheumatoid arthritis, reactive arthritis), systemic lupus erythematosus (SLE), psoriasis, inflammatory bowel disease (IBD) (including ulcerative colitis and Crohn's disease), encephalomyelitis, uveitis, myasthenia gravis, multiple sclerosis, insulin dependent diabetes, Addison's disease, celiac disease, chronic fatigue syndrome, autoimmune hepatitis, autoimmune alopecia, ankylosing spondylitis, fibromyalgia, pemphigus vulgaris, Sjogren's syndrome, Kawasaki's Disease, hyperthyroidism/Graves disease, hypothyroidism/Hashimoto's disease, endometriosis, scleroderma, pernicious anaemia, Goodpasture syndrome, Guillain-Barre syndrome, Wegener's disease, glomerulonephritis, aplastic anaemia (including multiply transfused aplastic anaemia patients), paroxysmal nocturnal hemoglobinuria, idiopathic thrombocytopenic purpura, autoimmune hemolytic anaemia, Evan's syndrome, Factor VIII inhibitor syndrome, systemic vasculitis, dermatomyositis, polymyositis and rheumatic fever, autoimmune lymphoproliferative syndrome (ALPS), autoimmune bullous pemphigoid, Parkinson's disease, sarcoidosis, vitiligo, primary biliary cirrhosis, and autoimmune myocarditis.

Organ transplant includes, but is not limited to, a liver transplant, a lung transplant, a kidney transplant, a heart transplant, a spleen transplant, a pancreas transplant, a skin transplant/graft, an intestine transplant, a cornea transplant and a thymus transplant.

The methods described herein can be used for determining a course of treatment for a patient in need thereof (i.e. a cancer patient). Cancer includes, but is not limited to, colorectal, hepatocellular, gallbladder, pancreatic, oesophageal, lung, breast, prostate, skin (i.e. melanoma), head and neck, renal cell carcinoma, ovarian, endometrial, cervical, bladder and urothelial cancer.

The methods described herein can be used to diagnose diseases of lymphoid cells (i.e. T lymphocytes and/or B lymphocytes, including cells of any developmental, differentiative or maturational stage of the lymphoid lineage of hematopoietic cells) such as lymphoid hematological malignancies or other lymphoproliferative disorders, and for detecting minimal residual disease (MRD) in subjects following treatment for such conditions.

Non-limiting examples of diseases of lymphoid cells for which the method described herein may usefully aid in diagnosis and/or MRD detection include lymphoid hematological malignancies such as acute lymphoblastic leukemia (ALL), multiple myeloma, plasmacytoma, macroglobulinemia, chronic lymphocytic leukemia (CLL), other lymphomas and leukemias including Hodgkins and non-Hodgkins lymphoma, cutaneous T-cell lymphoma, mantle cell lymphoma, peripheral T-cell lymphoma, hairy cell leukemia, T prolymphocytic lymphoma, angioimmunoblastic T-cell lymphoma, T lymphoblastic leukemia/lymphoma, peripheral T-cell lymphoma-not otherwise specified, adult T-cell leukemia/lymphoma, mycosis fungoides, Sezary syndrome, T lymphoblastic leukemia and any other cancer involving T-cells or B-cells; and may also include other lymphoproliferative disorders, including myeloproliferative neoplasms, myelodysplastic syndrome, and others.

The detection of MRD can play a significant role not only in monitoring a patient's response to therapy, but also in the accurate diagnosis of the underlying cause of major clinical signs. MRD typically refers to the presence of malignant cells (usually in reference to leukemic cells) that are not detectable on the basis of cellular morphology. Several studies have shown that quantitative detection of MRD in lymphoid malignancies predicts clinical outcome.(Bahloul et al., 2005; Bruggemann et al., 2004; Cave et al., 1998; Ciudad et al., 1999; Coustan-Smith et al., 2002; Coustan-Smith et al., 2000; Hoshino et al., 2004; Lucio et al., 1999; Radich et al., 1995; Szczepanski et al., 2001; van Dongen et al., 1998; Wells et al., 1998). The methods described herein can be used for diagnosis, for example, may include detecting MRD in lymphomas.

Monitoring the response of a cancer patient to a therapeutic treatment on the basis of tumour load quantification (i.e. by MRD detection) may assist in the assessment of a relative risk of relapse, and can also be used to identify patients who may benefit from therapy reduction, therapy intensification, reduction of immunosuppression for graft-versus-leukaemia effect after a stem cell transplant, or adoptive T-cell therapy (Bradfield et al., 2004). Minimal disease may also be encountered in diagnostic situations. For example, low levels of monoclonal B-cells in patients presenting clinically with cytopenia may raise suspicions for a diagnosis of myelodysplastic syndrome (Wells et al., 2003). Minimal disease detection is also encountered in staging of lymphoma, which may involve the detection of low levels of tumour cells against a background of normal cells. The detection of minimal disease as described herein (i.e. as MRD detection in lymphoid cancer patients following treatment) need not be limited to monitoring the effects of treatment, but may also find uses in diagnostic settings where no reference population is available for comparison.

Minimal residual disease can be detected by quantifying the adaptive immune cells from DNA extracted from a first sample obtained from a subject (i.e. bone marrow, lymph or blood, depending on the type of cancer) obtained using the methods described herein wherein the first sample is taken at a first time point before or during a therapeutic treatment, and wherein the first sample comprises a population of T- or B-cells. Subsequently, extracting from a second sample from the subject, wherein the second sample is taken at a later time point than the first sample, wherein the presence of T- or B-cells in the second sample indicates the presence of minimal residual disease.

The subject from which the sample is obtained may be known to have, or may be suspected of having or being at risk for having, a lymphoid hematopoietic cancer or other malignant condition, or an autoimmune disease, or an inflammatory condition. Alternatively, the subject from which the sample is obtained may be known to be free of a risk or presence of such disease.

For example, the subject is a patient that has been diagnosed as having or being at risk for developing or acquiring cancer according to art-accepted clinical diagnostic criteria, such as those of the U.S. National Cancer Institute (Bethesda, MD, USA) or as described in DeVita, Hellman, and Rosenberg's Cancer: Principles and Practice of Oncology (2008, Lippincott, Williams and Wilkins, Philadelphia/Ovid, New York); Pizzo and Poplack, Principles and Practice of Pediatric Oncology (Fourth edition, 2001 , Lippincott, Williams and Wilkins, Philadelphia/Ovid, New York); Vogelstein and Kinzler, The Genetic Basis of Human Cancer (Second edition, 2002, McGraw Hill Professional, New York); Dancey et al. (2009 Semin. Oncol. 36 Suppl.3:S46). Alternatively, the subject may be known to be free of a risk for having, developing or acquiring cancer by such criteria.

In some methods, two or more samples may be obtained from a single tissue (i.e. a single tumour tissue) and the relative representations of adaptive immune cells in the two or more samples are quantified to consider variations (e.g. heterogeneous infiltration) in different sections of a test tissue.

Information from the methods described herein will usefully provide information concerning the physiological and pathological status of a sample (and hence the subject from which the sample is derived), and will be particularly useful in situations where samples are obtained before, during and/or after therapy are assayed to quantify the adaptive immune cells. For instance, the amount of TILs in a tumour tissue may provide diagnostic and/or prognostic information, including information regarding the potential efficacy of a therapeutic regimen or regarding the optimal dosing regimen. Similarly, the amount of TILs in a tissue that is a target of autoimmune attack may usefully permit identification and refinement of clinical approaches to autoimmune disease.

Another example of the application of the methods described herein is monitoring treatment of T-cell lymphoma by B-cell depletion i.e. Rituximab. Reduction of infiltrated B-cell numbers would be the biomarker of choice to determine the treatment efficacy. The methods described herein provide a means for monitoring B-cell numbers in small tissue samples that do not require intact cells.

Autoimmune diseases like rheumatoid arthritis are in part B-cell driven and can be diagnosed by determining the number of B-cells. Moreover, treatment options nowadays also include B-cell depleting agents like Rituximab. Before entering such treatment modalities and during treatment follow-up, the presence of B-cells should be monitored. Compared to the current lymphocyte monitoring approaches, DNA-based quantifications such as the methods described herein offer a less invasive method of monitoring. Mostly because of the minimal requirements of the DNA samples, less invasive sampling methods can be applied. This is of particular benefit to subjects like children with immune deficiencies that require repeated monitoring. The methods described herein reduce invasiveness of biopsies and is thus important for reducing disease burden, especially in children.

Specific examples of practical applications of the methods are described herein. For example, for disease of the eye (i.e. uveitis or B-cell lymphoma); the eye fluid is normally considered cell-free, however the methods described herein can be used to measure B and T-cell counts for the diagnosis of diseases such as uveitis or B-cell lymphoma. Inflammation in the eye (uveitis) is correlated with high T-cell numbers. Moreover, lymphoma present an excess of B-cells. The methods described herein can therefore be used for diagnosis of said diseases by quantification of adaptive immune cells in an eye derived sample.

### Kits

Kits are also provided herein for use in the methods of the invention. The kits may comprise primers and/or probes for specifically amplifying the markers and regional correctors mentioned in the methods. The kit may also comprise a thermostable polymerase and/or labeled dNTPs or analogs thereof. The labeled dNTPs or analogs thereof may be fluorescently labeled. The kit may comprise, as well as the primers and/or probes, reagents necessary for carrying out the methods of the invention, for example enzymes, dNTP mixes, buffers, PCR reaction mixes, chelating agents and/or nuclease-free water. The kit may comprise instructions for carrying out a method of the invention. Moreover, the kits may be provided with dedicated software that enables optimal analysis of cell counts.

Aspects of the invention are demonstrated by the following non-limiting examples.

### EXAMPLES

### Example 1: T-Cell Quantification

### Genomic Instability

An ongoing challenge is the selection of stable reference genes in different types of malignant samples or other samples with copy number variation and/or potentially unstable DNA copy numbers. When copy number alterations affecting the reference are present in a DNA sample, incorrect quantifications are obtained. Therefore, choosing a stable reference is needed to obtain optimal results. By analysing multiple reference genes, genomic aberrations can be recognized and corrected for.

Duplex and Multiplex experimental Setup

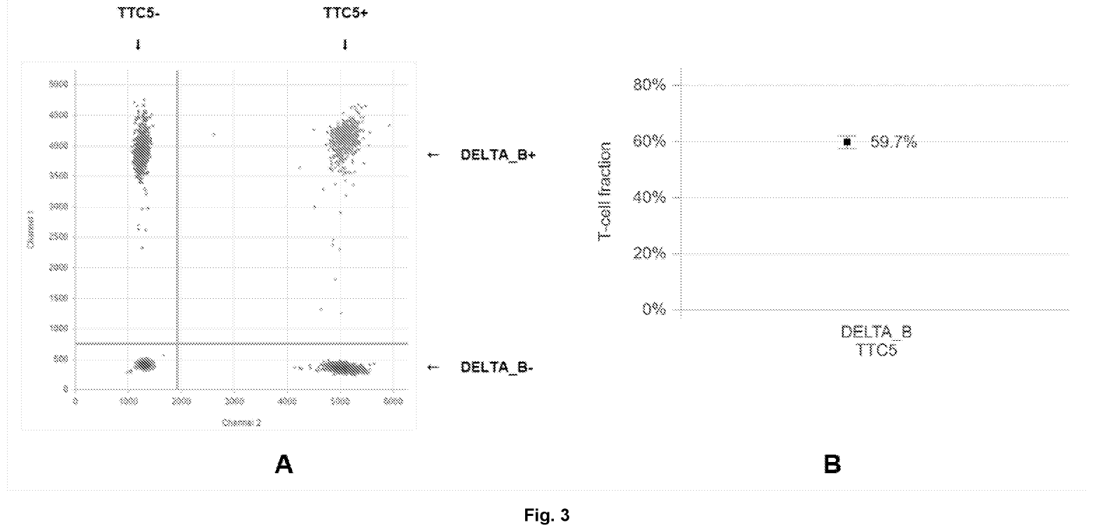

Digital PCR experiments are typically carried out in a duplex setup; in which one target on fluorescence channel 1 (FAM) and one target on fluorescence channel 2 (HEX) are measured simultaneously. Following this setup, a totally new approach to quantify T-cell presence in DNA samples has been introduced and validated by the inventors (Zoutman et al., 2017). In short, a ΔB T-cell marker target and a diploid reference DNA marker was measured in one digital PCR experiment, which required only 5-50 ng of DNA (FIGS. 3A and 3B). Using the same principles, other immune cell (sub)populations could also be identified and quantified.

The inventors have also performed immune cell quantifications in a multiplex setup. This setup requires the same amount of DNA as a traditional duplex experiment (Hughesman et al., 2017), but gives more information as up to two additional targets may be analysed within the same experiment.

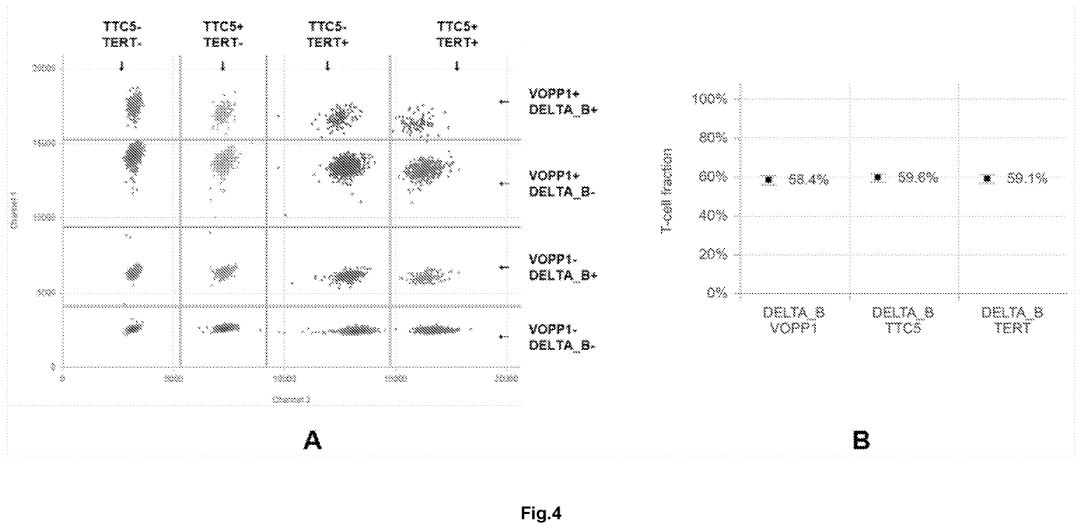

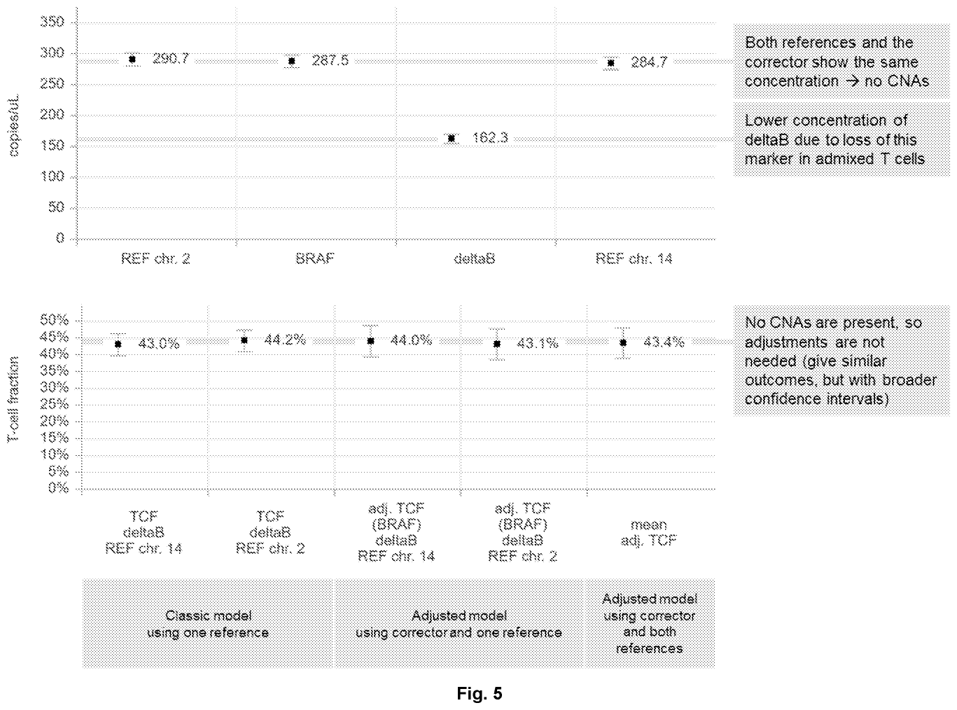

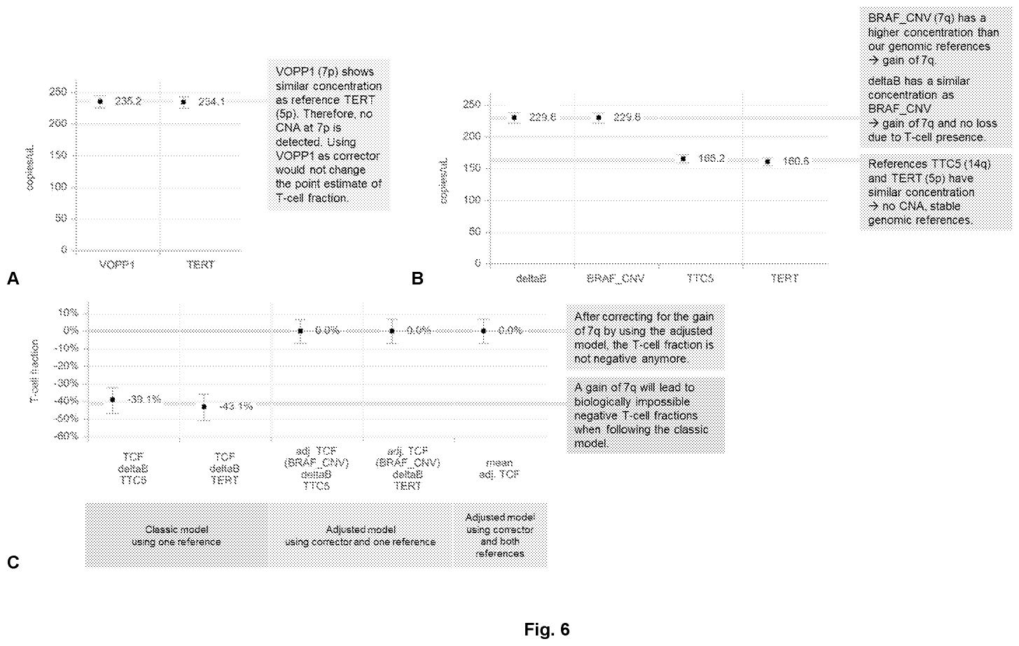

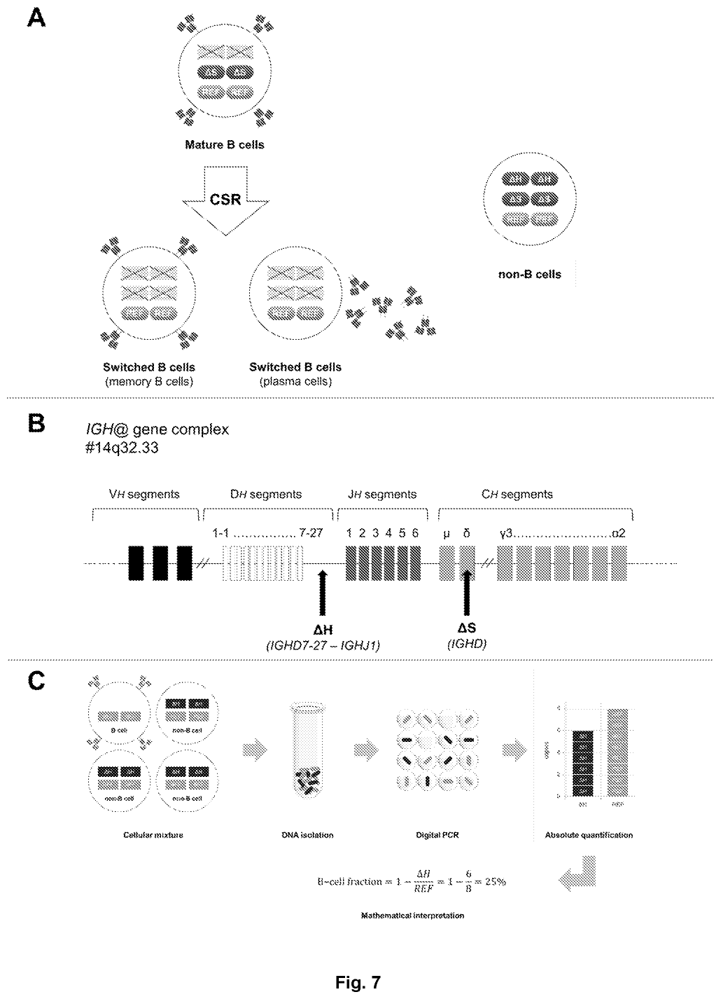

An example of a multiplex setup is given in FIGS. 4A and 4B, in which two additional references are added to the analysis. This setup enables a more accurate quantification with a limited amount of DNA (<25 ng) as the stability of the chosen references can be analysed within the same experiment.

### Analysing Non-Malignant Samples

When analysing a known non-malignant sample (large majority of clinical samples), such as a healthy PBMC, or a biopsy from a rheumatoid arthritis patient, no copy number alterations at any of the chromosomes are to be expected and indeed none were observed (FIG. 5). In these instances, the classical model for such samples may be used, as this provides accurate T-cell quantifications (Zoutman et al., 2017). By using the adjusted model, there was no change in the outcome, i.e. the point estimate of T-cell fraction.

In non-malignant samples, genomic instability may be less common and one reference may be sufficient to calculate the presence of a specific type of immune cells. In these samples, multiple marker assays could be combined in a multiplex setup, by which several immune cell (sub)populations could be quantified within one experiment. This would be particularly useful for samples of limited quantity, such as small biopsies.

### Analysing Samples With CNV, Such as Malignant Samples

When a malignant sample is analysed, a researcher should always be aware of possible copy number alterations involving any of the informative genomic regions. Importantly, as each malignant sample may have its own unexpected alterations, only general recommendations on how to test such specimens are described herein. The inventors described some of these recommendations in their earlier publications (Zoutman et al., 2019; Zoutman et al., 2017).

Two possible problems may be encountered. The first is that copy number instability may be present at the ΔB or ΔD locus. The second problem is that copy number instability may be present at the reference loci. The consequence of using the classical model would lead to a T-cell fraction determination that is underestimated or overestimated.

One solution is to switch to the other assay, for example use ΔD when only the locus of ΔB is altered or use ΔB when only the locus of ΔD is altered.

The second solution to this problem is to use the adjusted model. When a copy number alteration (CNA) involving the ΔB or ΔD T-cell marker locus is present, the adjusted model can be used to correct for this alteration. Mathematically, the inventors use the corrector to adjust for CNAs involving the TRB locus. The corrector assay was therefore able to exactly indicate the copy number of this locus.

In de Lange et al. 2018 an assay for chromosome 7p (VOPP1) was used, and it was assumed any CNAs involving ΔB (chromosome 7q) would equally involve VOPP1. However, due to the large distance between these two markers (they are on different arms of the chromosome), more regional CNAs involving the ΔB locus were not covered by measuring VOPP1.

A demonstration of this phenomenon is given in a tumour (uveal melanoma) sample (FIG. 6), in which no alteration at VOPP1 was detected (chromosome 7p is stable), but a chromosome 7q gain resulted in negative T-cell fractions, which is biologically impossible. Other molecular characteristics of this tumour sample suggested a very low to absent infiltration of T-cells. VOPP1 and the reference marker TERT (chromosome 5p) concentrations were comparable in the sample. Therefore, no CNAs were accounted for when determining T-cell fraction via ΔB if VOPP1 was used as the regional corrector. In contrast, using BRAF (a gene located on the 7q arm like ΔB), the gain was recognized and the adjusted model was used. Thereby by using a more local regional corrector (BRAF), a non-negative, biologically plausible T-cell fraction of 0% was calculated (FIG. 6).

### Example 2: B-Cell Quantification

B-cells fulfill an important role in the adaptive cell-mediated immunity. Moreover, upon activation, most B-cells function in the humoral immunity compartment as plasma cells by secreting antibodies. For clinical applications, it can be important to quantify B-cells accurately in a variety of body fluids and tissues of benign, inflammatory or malignant origin. For decades, flow cytometry and immunohistochemistry have been the accustomed methods to quantify B-cells. Although these methods are widely appreciated, they depend on the accessibility of B-cell epitopes and therefore require fresh, frozen or fixed material of a good quality. Whenever samples are low in quantity and/or quality, an accurate quantification can be difficult. By shifting the focus from epitopes to DNA markers, quantification of B-cells remains achievable.

Protein cell surface markers are expressed in a large variety of levels. Despite this, they are very useful to identify and quantify specific cell types, provided that the cellular context remains intact. Once this context is lost, these molecules are not representing the actual number of originating cells anymore. In contrast to translationally and transcriptionally expressed molecules, genomic DNA is normally present in equal (diploid) amounts per cell. Once the cellular context is lost, DNA molecules (e.g. in solution) still represent the actual number of originating cells. Simply put, DNA molecules relate to the number of cells in a more digital manner as compared to varying (analogue) numbers of expressed molecules.

Unfortunately, in benign situations, the availability of different cell type-specific DNA markers is very limited. Consequently, it is challenging to develop a DNA based-method to quantify specific cell types. However, an exception are B-cells. During cell development, B-cells are subjected to programmed genetic recombination processes which will result in deletion of specific sequences in the IGH@ locus. These cell type-specific DNA “scars” (loss of sequences) can be exploited as B-cell markers. Even without cellular context, presence or absence of these scars relates to the actual number of B-cells in a digital way, respectively. This type of (digital) cell-specific markers can be counted by a corresponding digital technique of quantification, e.g. digital PCR.

Here, the inventors describe a simple and sensitive digital PCR-based method to quantify B-cells relatively fast, accurately and independently of the cellular context, offering new possibilities for quantification for example in small volume samples and samples with a low DNA concentration, like liquid biopsies.

Since B-cells play an important role in the adaptive cell-mediated and humoral immunity, quantifying these lymphocytes accurately in benign, inflammatory and malignant tissues or body fluids can be of great importance in a variety of clinical management.

For instance, quantifying B-cells in benign and (chronic) inflammatory diseases can be valuable in terms of diagnostics. In autoimmune diseases like arthritis the fraction of B-cells is commonly ascertained to monitor disease progression. Furthermore, since directed B-cell eradication is one of the treatment modalities, monitoring treatment efficacy by accurate B-cell quantification is warranted (Costa et al., 2016).

With respect to malignancies, the magnitude of T-cell infiltration has been correlated both positively and negatively to tumour growth and clinical prognosis, but the role of B-cells is underestimated (Castaneda et al., 2016; Fridman et al., 2011; Schatton et al., 2014; Talmadge, 2011). Increasing evidence supports a correlation between B-cell infiltration and clinical prognosis and prediction to therapy response (Linnebacher and Maletzki, 2012; Shen et al., 2018). Furthermore, some studies associate B-cell infiltration with an impaired immune response. Thereby, eradication of the B-cell compartment has also been suggested as therapy to improve anti-tumour response (Schwartz et al., 2016; Theurich et al., 2016). Hence, accurate quantification of B-cells is valuable and of great importance in view of many clinical aspects.

Accustomed quantification methods to determine B-cell content in body fluids or solid tissues are flow cytometry and immunohistochemistry. These methods are very precise through the use of cell-specific antibodies, e.g. directed against CD19 or CD20 for B-cells and CD38 for plasma cells. Accordingly, availability of these markers and access to the associated epitopes are required. Presence and accessibility are mainly related to the specimen's condition and applied preparation method. Mostly, fresh, frozen and fixed material meet the demanded criteria for an accurate quantification of B-cells (Walker, 2006; Wood et al., 2013). Whenever sample quantity and/or quality is too low (loss of cellular context), quantification can be impeded. Alternatively, the method of quantification may be shifted from a focus on epitope expression to cell-specific DNA markers.

Conventionally, multiplex PCR, combined with deep sequencing techniques, can be applied to determine B-cell content on a genomic level. However, these approaches typically require an amplification step, thereby limiting possibilities for absolute quantification and allowing merely for interpretation of relative differences. Moreover, these approaches target the whole repertoire of immunoglobulin (IG) genes and thereby supplying additional information about gene use (Carlson et al., 2013; Evans et al., 2007; van Dongen et al., 2003). Consequently, a simple B-cell quantification results into a complex, expensive and time-consuming procedure. To avoid to this, the inventors took advantage of the generic dissimilarity between B-cells and cells of other origin by measuring loss of specific germline IGH@ loci to quantify VDJ rearranged B-cells and in particular switched B-cells. Thus, instead of counting a whole repertoire of rearranged IG genes, the inventors designed an indirect counting approach based on the same rationale as previously published for the quantification of T-cells (Zoutman et al., 2019; Zoutman et al., 2017).

In contrast to the varying (analogue) numbers of expressed surface molecules, all somatic nucleated cells contain genomic DNA in equal (diploid) amounts. Hence, even without a cellular context, there is a direct correlation between the amount of molecules in DNA specimens and the number of originating cells. So to speak, presence or absence of genomic DNA correlates directly to the number of originating cells in a digital-like way. In addition, whereas epitopes may vary in expression between different cell populations under physiological conditions (e.g. aging), DNA content of cells always remains in a diploid conformation (Ginaldi et al., 2001). Consequently, DNA markers have a high quantitative potential, especially when epitope-based approaches are not feasible.

On the other hand, whereas a broad variety of transcriptionally and translationally expressed cell-type specific molecules has been identified, the DNA sequence is essentially the same in all cells, thereby barely providing cell-type specific markers. However, during maturation and differentiation, B-cells are subjected to programmed genetic recombination processes, like VDJ rearrangements and class switch recombination (CSR). These recombination processes result in deletion of specific sequences of the IGH@ locus in B-cells and switched B-cells specifically. The inventors regard these specific scars (loss of dedicated IGH@ sequences) as cell markers for B-cells. Therefore, even without cellular context, presence or absence of these markers relates directly to the actual number of B-cells in a digital way.

Immunoglobulins (IGs) are antigen-binding molecules which are translationally expressed by VDJ rearranged B-cells. Initially, peripheral VDJ rearranged naïve B-cells express non-autoreactive IGs predominantly as surface membrane-bound molecules. Subsequently, upon activation, initiated by an encounter with a complementary antigen, B-cells migrate to secondary lymphoid organs for further diversification of the IGs. In the dark zone of germinal centers, clonal expansion, in combination with somatic hypermutation, will result in B-cell clones (centroblasts) with a changed affinity to its activating antigen. Thereafter, in the light zone of germinal centers, most B-cell clones (centrocytes) with an improved affinity will further differentiate into IG-secreting plasma cells, while a smaller fraction will differentiate into memory B-cells. Genetically, this second differentiation is conducted by CSR, wherein the constant region of IGs is replaced by another downstream constant region without altering antigen specificity of the original IG (FIG. 7) (Gonzalez et al., 2007; Liu et al., 1996; Ollila and Vihinen, 2005; van Dongen et al., 2003).

Surface membrane-bound and secreted IGs are translationally expressed as complex heterodimers consisting of two identical heavy chains encoded by the IGH@ gene cluster and two identical light chains encoded by either the kappa (IGK@) or lambda (IGL@) gene clusters. The IG repertoire in the periphery is highly diverse, supporting the capability to recognize many different epitopes of harmful antigens and pathogens. Similar to T-cell receptors, the basis of diversity lies in the programmed combinatorial rearrangement of VDJ genes during early B-cell maturation in the bone marrow. Throughout lymphoid differentiation, many distinct variable, (diversity) and joining IG genes are rearranged. Ultimately, these rearrangements, followed by other DNA sequence altering mechanisms like junctional diversity and combinatorial association of translated heavy and light chains, result in a highly diverse repertoire of antigenic IGs (Gonzalez et al., 2007; Liu et al., 1996; Ollila and Vihinen, 2005; van Dongen et al., 2003).

As compared to the light chain encoding genes (IGK@ and IGL@), IGH@ is ubiquitously expressed by all VDJ rearranged B-cells. Also on genetic level, this gene is rearranged first in the cascade of sequentially executed recombination of IG genes during B-cell development. Considering this, IGH@ is highly suitable to be exploited for quantification of VDJ rearranged B-cells. During IGH@ rearrangements allelic exclusion is applied to prevent heterozygous expression of both processed alleles. Since VDJ rearrangements are extremely error-prone, depending on the productivity of a rearranged gene, the other allele is often processed in VDJ rearranged B-cells as well (Vettermann and Schlissel, 2010). However, some parts of the IGH@ gene are rearranged biallelically regardless of allelic exclusion and can consequently be used as genomic B-cell marker. For instance, the intergenic sequence between genes IGHD7-27 and IGHJ1 (IGH@ at 14q32.33) is lost biallelically in virtually all VDJ rearranged B-cells; the inventors called this region ΔH (FIG. 7A-B) (Lefranc, 2001). By measuring the exact loss of this ΔH locus in DNA specimens, it is possible to determine the contribution of B-cells among other cell types in a quantitative manner.

To quantify switched B-cells specifically, the inventors exploited another genomic recombination process (CSR) which takes place in activated B-cells once migrated to germinal centers. During CSR, the IGH@ gene cluster undergoes a second recombination process by which the constant genes IGHM and IGHD become deleted and replaced by another downstream gene (IGHG3, IGHG1, IGHA1, IGHG2, IGHG4, IGHE or IGHA2). This recombination of constant domains results in memory B-cells, but mostly in IG-secreting plasma cells, producing IgG3, IgG1, IgA1, IgG2, IgG4, IgE or IgA2 antibodies correspondently. Thus, as a consequence of CSR, IGHM and IGHD genes (IGH@ at 14q32.33) are lost in all switched (plasma and memory) B-cells. The inventors designed an assay on IGHD and called this region ΔS (FIG. 7A-B) (Lefranc, 2001; Liu et al., 1996; Vettermann and Schlissel, 2010). By measuring the exact loss of this ΔS locus in DNA specimens, it is possible to determine the contribution of switched B-cells specifically among other cell types in a quantitative manner. Generally, it is accepted that CSR only occurs on the active IGH@-allele. However, circumstantial evidence suggests involvement of the non-productive allele as well (Pichugin et al., 2017). Based on the data generated herein, the inventors have identified ΔS as a means for distinguishing between switched and non-switched B-cell populations within a sample.

Mathematically, it is possible to calculate the fraction of non-switched B-cells whenever loss of ΔH and ΔS has been quantified. By subtracting the determined number of switched B-cells from the enumerated VDJ rearranged B-cells, the remaining fraction of non-switched VDJ rearranged B-cells can be calculated.

As stated before, DNA molecules relate to the number of originating cells in a more digital way as compared to varying (analogue) numbers of expressed molecules. Consequently, presence or absence of marker ΔH and ΔS directly relates to the number of VDJ rearranged, switched and non-B-cells likewise. These B-cell specific DNA markers can be quantified by a corresponding digital technique like digital PCR. This method enables sensitive, precise and reproducible absolute quantification of nucleic acids by combining sample partitioning (limiting dilution) with Poisson statistical data analysis (Vogelstein and Kinzler, 1999). When partitioning samples, admixed nucleic acid molecules and PCR solution are separated into thousands of partitions (e.g. droplets) prior to PCR amplification (FIG. 7C). After amplification with specific primers and fluorescently labeled hydrolysis probes, droplets with and without PCR products are counted by measuring fluorescence intensities. By applying the statistics of Poisson distribution on scored droplets an absolute measurement of nucleic acid concentration can be achieved. Regarding the determination of copy number variations (CNVs) in DNA specimens, a multiplex amplification of target and reference sequences can be carried out using (differently) mixed and labeled hydrolysis probes (e.g. with 6-Carboxyfluorescein (FAM) and hexachlorofluorescein (HEX) dyes). By comparing target and reference quantifications within the same experiment, multiple sources of variation are excluded and gains and losses in DNA content can be determined accurately (Whale et al., 2016).

To determine the B-cell content in DNA specimens by using digital PCR, the inventors developed an approach which is basically similar to conventional CNV measurements. Since loss of germline IGH@ loci ΔH and ΔS is uniquely related to the number of VDJ rearranged and switched B-cells, an accurate quantification of these cells can be obtained by measuring loss of these targets, relative to a copy number stable reference gene (FIG. 7C).

Under benign conditions, copy number instability may not be expected and any reference would suffice. When copy number instability might affect the IGH@ locus, a regional corrector target is needed to recognize and normalize genetic imbalances. Optimally, this regional corrector is as close as possible to the IGH@ gene cluster without being affected by VDJ rearrangements or CSR. Regarding this, the most appropriate and proximal candidate gene is IGHA2, the last downstream constant gene of the IGH@ cluster.

In conclusion, the inventors designed an accurate, sensitive and relative fast method to quantify VDJ rearranged, switched and non-switched B-cells specifically in DNA specimens. This digital PCR approach is less devious, expensive and time-consuming as compared to multiplex PCR and deep sequencing techniques wherein the full repertoire of recombined IG genes is amplified and quantified. Moreover, an absolute and direct quantification of all VDJ rearranged and switched B-cells, as performed by digital PCR, is less biased because of its digital design. Other methods, like flow cytometry, immunohistochemistry and (multiplex) PCR techniques are more vulnerable to bias in quantification due to arbitrary aspects like instrument settings, dependency on standards and replicates and even personal factors (Bustin et al., 2009; Robins et al., 2013; Walker, 2006; Wood et al., 2013) (Vogelstein and Kinzler, 1999).

Importantly, the sample requirements are much lower compared to cell-based methods, as no intact cells or preserved epitope expression are needed. Instead, reliable quantification can be obtained from several nanograms of DNA, correspondently offering new possibilities for quantification in small volume samples, like liquid biopsies.

Validation of the inventors' genetic B-cell assays as well as the switched B-cell assay has also been performed based on a B-cell pool (BCP) and a B-cell line L363 mixed with fibroblast DNA in order to generate dilution curves (FIG. 8). The dilutions of both BCP and L363 are combined to provide extensive coverage.

### Example 3: ΔH is a B-Cell Specific DNA Marker and is Biallelically Lost

Specificity of the designated mature B cell DNA marker was evaluated by measuring loss of ΔH in a variety of (sorted) samples. Since ΔH is lost due to the first VDJ rearrangement of the IGH@ locus during early B-cell development (i.e DH-JH rearrangement), this marker should be specifically lost in DNA samples of B-cell origin.

Initially, the presence and absence of ΔH was assessed by using dPCR in DNA specimens from L-363 and fibroblast cell cultures which served as pure sources of B-cells and non-B cells respectively. The inventors observed no loss of ΔH in fibroblasts, however, in L-363 cells this marker was undetectable, indicating biallelic loss of this part of the IGH@ locus.

To confirm this finding in benign and uncultured B-cells, the inventors tested a variety of B-cell subpopulations from healthy blood donors. These samples were obtained by FACS using diverse gating strategies: In 3 donors they tested two different populations of mature unswitched B cells; Both IgD and IgM expressing B cells were analyzed separately. Besides using unswitched B-cells, they also sorted switched B cells using three different gating strategies. The inventors sorted both IgA and IgG memory B cells separately from 2 donors. In addition, for 2 other donors, they applied an indirect gating strategy, in which they excluded unswitched B cells by gating for CD27+IgD-CD19+ (CD24HIGH and CD38HIGH excluded). By using dPCR and presuming a biallelic loss of ΔH in mature B cells, the inventors quantified the B-cell purity in DNA specimens from the differently FACS sorted cells. In 11 out of 12 samples the B-cell purity was >99% and in the other sample it was >98% (see FIG. 10).

These results demonstrate efficiently executed cell sorting of B-cell subpopulations and, moreover, reaffirmed that marker ΔH is specifically, ubiquitously and biallelically lost in mature (unswitched and switched) B cells.

### Example 4: ΔS is a Switched B-Cell Specific DNA Marker and is Often Biallelically Lost

To evaluate the specificity of the switched B cell DNA marker ΔS, the inventors used the same samples as described above. Since ΔS is lost as a result of CSR, this marker should be lost in DNA samples from switched B-cells specifically.

Initially, when analyzing cell cultures, the inventors observed no loss of ΔS in fibroblasts. However, it was completely lost in L-363 cells, reflecting biallelic loss of IGHD. Since L-363 cells are derived from leukemic IgG plasma cells and are clonally expanded, observed biallelic loss of the ΔS locus may not be a generic event in other (benign) switched B cell specimens accordingly.

To investigate to what extent biallelic loss of ΔS occurs in benign, uncultured and polyclonal B-cell samples, the inventors used the same 6 unswitched and 6 switched sorted B-cell samples as mentioned above. Since unswitched B cells did not undergo CSR, ΔS should still be present in these cells. Conversely, switched B cells undergo CSR and therefore will lose ΔS. When analyzing unswitched B cell DNA samples by dPCR, the inventors observed no loss of the ΔS marker in any of the samples. Previously, these samples were identified as true mature B cell populations by the observation of complete (biallelic) loss of ΔH. By adding the obtained knowledge of intact ΔS loci, the inventors are assured that these cells did not undergo CSR and consequently are true unswitched B cells as well. In contrast, ΔS was equivalently and consistently lost in on average 81% (i.e. a fraction of 0.81) of the measured alleles in DNA specimens from switched B cells (see FIG. 11). Presuming that CSR at least occurs with one allele, the inventors can extrapolate that in approximately ⅗ of the switched B cells ΔS was biallelically lost and in the remaining fraction monoallelically.

### Example 5: An Allelic-Factor (AF) Corrects Biological Imbalances

ΔS is not ubiquitously lost in either a pure mono- or biallelic fashion in benign, uncultured and polyclonal B-cell samples. By adding an allelic-factor (AF) to the formula for quantification of switched B cells, the inventors are able to enumerate these cells accurately in DNA samples by using dPCR.

The formula to quantify switched B-cells was:

[switched B cells]=(1−[dS]/[Ref])

By adding an allelic-factor (AF), the formula turns into:

[switched B cells]=(1−[dS]/[Ref])/AF

To apply this formula on the cohort of samples as described above, the formula will be:

[switched B cells]=(1−[dS]/[Ref])/0.81

These results demonstrate that the ΔS locus is a specific switched B-cell marker and can be used to quantify this cell type in DNA specimens. Moreover, the inventors demonstrated that ΔS is often lost biallelically in switched B cells, and by adding an allelic-factor (AF) to the formula to quantify these cells in DNA samples, they can effectively correct for biological imbalances.

### Example 6: Pan-Cancer Analysis of Copy Number Alterations (CNA's) Affecting the T- and B-Cell Marker Loci

To investigate the frequency and origin of CNA's affecting the ΔD and ΔB T-cell marker regions, and ΔH and ΔS B-cell marker region in cancer, the inventors analysed the copy number profiles of 10,522 cases spanning 31 tumour types from the TCGA pan-cancer dataset (Cancer Genome Atlas Research, N., et al, 2013; Taylor et al., 2018). On average ˜17% of the specimens carried a CNA affecting the TRD gene (in which the ΔD marker is located), ˜24% affecting the TRB gene (in which the ΔB marker is located) and ˜21% affecting the IGH gene (in which the ΔH and ΔS markers are located). However, large differences were observed between cancer types (FIGS. 12 to 14). Still, these frequencies indicate that classical DNA-based T- and B-cell quantification will fail in on average ¼ (ΔB) or ⅙ (ΔD) or ⅕ (ΔH and ΔS) of the cancer specimens, marking the need for a robust solution: the adjusted model described herein.

The T- and B-cell marker regions are not necessarily the target of these CNA's. For example, ΔB gains and losses originated from complete chromosome 7 alterations in half of the affected cases (FIG. 15A). The more focal CNA's were typically gains and co-involved BRAF, an established oncogene located only 1.35 million base pairs from the TRB gene. Similarly, CNA's affecting the TRD or IGH gene frequently originated from the loss or gain of an entire chromosome 14 (FIG. 15B-C). However, to be able to correctly recognise and normalise these alterations in all cases (i.e. also those cases with a focal CNA), a regional corrector as close as possible to ΔD or ΔH and ΔS respectively remains recommended.

### Example 7: Validation of Multiplex Digital PCR Setup for ΔB Adjusted Model

To evaluate the wet-lab performance of the adjusted model, the inventors designed a multiplex digital PCR setup analysing three targets (FIG. 16A):
T-cell marker ΔB (located in the TRB gene, assay on channel 2 with the lowest fluorescence);
regional corrector RC ΔB (located in the TRBC2 gene segment, the only assay on channel 1);
stable genomic reference REF (located in the TTC5 gene, assay on channel 2 with the highest fluorescence).

As digital PCR partitions may or may not contain each of the three targets, 2\hat{ }3=8 different clusters can appear in the 2D space. When these clusters are clearly distinct, all three targets can be measured concurrently and the T-cell fraction according to the adjusted model can be calculated. Notably, as ΔB and REF are both being determined in this multiplex experiment, the T-cell fraction according to the classic model can also be calculated.

As a first practical validation, the inventors analysed a healthy PBMC DNA sample and compared the target concentration ratios obtained with this multiplex setup with those from two separate duplex experiments (FIG. 16B). Very similar ratios were observed with both setups, but the multiplex approach required only half of the amount input DNA and experimental reagents. This demonstrates that the multiplex experiments are equally valid, but more efficient than the traditional duplex setups.

To study the performance of the approach described herein in copy number stable samples, the inventors applied the multiplex setup in 6 healthy PBMC samples for which flow cytometry-based T-cell quantifications were available (FIG. 16C). Following both the classic and adjusted model, accurate T-cell fractions were obtained with high correlations to the fractions measured by flow cytometry (R=0.97-0.98).

Next, the inventors evaluated the approach in copy number unstable DNA mixtures of a healthy PBMC sample (60% T cells based on flow cytometry) and uveal melanoma cell line Mel-202 (0% T cells). This cell line harbours a gain of the entire chromosome 7, encompassing the ΔB T-cell marker region described herein (Nareyeck et al., 2006). Therefore, these mixtures are not correctly measurable following the classic model, similar to the situation proposed in FIG. 1—Adjusted model. Using the multiplex digital PCR experimental setup described herein, five of such mixtures were analysed (FIG. 16D). As expected, the more Mel-202 DNA present, the larger the error in T-cell fraction according to the classic model. Though, under all conditions the adjusted model allowed to correctly indicate the fraction of admixed T-cell DNA. This forms the in-vitro validation of the experimental rationale described herein.

### REFERENCES

Bahloul, M., Asnafi, V., and Macintyre, E. (2005). Clinical impact of molecular diagnostics in low-grade lymphoma. Best practice & research Clinical haematology 18, 97-111.
Bradfield, S. M., Radich, J. P., and Loken, M. R. (2004). Graft-versus-leukemia effect in acute lymphoblastic leukemia: the importance of tumor burden and early detection. Leukemia 18, 1156-1158.
Bruggemann, M., Pott, C., Ritgen, M., and Kneba, M. (2004). Significance of minimal residual disease in lymphoid malignancies. Acta haematologica 112, 111-119.
Bustin, S. A., Benes, V., Garson, J. A., Hellemans, J., Huggett, J., Kubista, M., Mueller, R., Nolan, T., Pfaffl, M. W., Shipley, G. L., et al. (2009). The MIQE guidelines: minimum information for publication of quantitative real-time PCR experiments. Clin Chem 55, 611-622.
Carlson, C. S., Emerson, R. O., Sherwood, A. M., Desmarais, C., Chung, M. W., Parsons, J. M., Steen, M. S., LaMadrid-Herrmannsfeldt, M. A., Williamson, D. W., Livingston, R. J., et al. (2013). Using synthetic templates to design an unbiased multiplex PCR assay. Nature communications 4, 2680.
Carter, S. L., Cibulskis, K., Heiman, E., McKenna, A., Shen, H., Zack, T., Laird, P. W., Onofrio, R. C., Winckler, W., Weir, B. A., et al. (2012). Absolute quantification of somatic DNA alterations in human cancer. Nat Biotechnol 30, 413-421.
Castaneda, C. A., Mittendorf, E., Casavilca, S., Wu, Y., Castillo, M., Arboleda, P., Nunez, T., Guerra, H., Barrionuevo, C., Dolores-Cerna, K., et al. (2016). Tumor infiltrating lymphocytes in triple negative breast cancer receiving neoadjuvant chemotherapy. World J Clin Oncol 7, 387-394.
Cave, H., van der Werff ten Bosch, J., Suciu, S., Guidal, C., Waterkeyn, C., Otten, J., Bakkus, M., Thielemans, K., Grandchamp, B., and Vilmer, E. (1998). Clinical significance of minimal residual disease in childhood acute lymphoblastic leukemia. European Organization for Research and Treatment of Cancer--Childhood Leukemia Cooperative Group. N Engl J Med 339, 591-598.
Ciudad, J., San Miguel, J. F., Lopez-Berges, M. C., Garcia Marcos, M. A., Gonzalez, M., Vazquez, L., del Canizo, M. C., Lopez, A., Van Dongen, J. J., and Orfao, A. (1999). Detection of abnormalities in B-cell differentiation pattern is a useful tool to predict relapse in precursor-B-ALL. Br J Haematol 104, 695-705.
Costa, S., Schutz, S., Cornec, D., Uguen, A., Quintin-Roue, I., Lesourd, A., Berthelot, J. M., Hachulla, E., Hatron, P. Y., Goeb, V., et al. (2016). B-cell and T-cell quantification in minor salivary glands in primary Sjogren's syndrome: development and validation of a pixel-based digital procedure. Arthritis research & therapy 18, 21.
Coustan-Smith, E., Sancho, J., Behm, F. G., Hancock, M. L., Razzouk, B. I., Ribeiro, R. C., Rivera, G. K., Rubnitz, J. E., Sandlund, J. T., Pui, C. H., and Campana, D. (2002). Prognostic importance of measuring early clearance of leukemic cells by flow cytometry in childhood acute lymphoblastic leukemia. Blood 100, 52-58.
Coustan-Smith, E., Sancho, J., Hancock, M. L., Boyett, J. M., Behm, F. G., Raimondi, S. C., Sandlund, J. T., Rivera, G. K., Rubnitz, J. E., Ribeiro, R. C., et al. (2000). Clinical importance of minimal residual disease in childhood acute lymphoblastic leukemia. Blood 96, 2691-2696.
Davis, M. M., and Bjorkman, P. J. (1988). T-cell antigen receptor genes and T-cell recognition. Nature 334, 395-402.
de Lange, M. J., Nell, R. J., Lalai, R. N., Versluis, M., Jordanova, E. S., Luyten, G. P. M., Jager, M. J., van der Burg, S. H., Zoutman, W. H., van Hall, T., and van der Velden, P. A. (2018). Digital PCR-Based T-cell Quantification-Assisted Deconvolution of the Microenvironment Reveals that Activated Macrophages Drive Tumor Inflammation in Uveal Melanoma. Mol Cancer Res 16, 1902-1911.
Dik, W. A., Pike-Overzet, K., Weerkamp, F., de Ridder, D., de Haas, E. F., Baert, M. R., van der Spek, P., Koster, E. E., Reinders, M. J., van Dongen, J. J., et al. (2005). New insights on human T cell development by quantitative T cell receptor gene rearrangement studies and gene expression profiling. The Journal of experimental medicine 201, 1715-1723.
Dube, S., Qin, J., and Ramakrishnan, R. (2008). Mathematical analysis of copy number variation in a DNA sample using digital PCR on a nanofluidic device. PloS one 3, e2876.
Evans, P. A., Pott, C., Groenen, P. J., Salles, G., Davi, F., Berger, F., Garcia, J. F., van Krieken, J. H., Pals, S., Kluin, P., et al. (2007). Significantly improved PCR-based clonality testing in B-cell malignancies by use of multiple immunoglobulin gene targets. Report of the BIOMED-2 Concerted Action BHM4-CT98-3936. Leukemia 21, 207-214.
Fridman, W. H., Galon, J., Pages, F., Tartour, E., Sautes-Fridman, C., and Kroemer, G. (2011). Prognostic and predictive impact of intra- and peritumoral immune infiltrates. Cancer Res 71, 5601-5605.
Ginaldi, L., De Martinis, M., D′Ostilio, A., Marini, L., Loreto, F., Modesti, M., and Quaglino, D. (2001). Changes in the expression of surface receptors on lymphocyte subsets in the elderly: quantitative flow cytometric analysis. Am J Hematol 67, 63-72.
Gonzalez, D., van der Burg, M., Garcia-Sanz, R., Fenton, J. A., Langerak, A. W., Gonzalez, M., van Dongen, J. J., San Miguel, J. F., and Morgan, G. J. (2007). Immunoglobulin gene rearrangements and the pathogenesis of multiple myeloma. Blood 110, 3112-3121.
Hoshino, A., Funato, T., Munakata, Y., Ishii, T., Abe, S., Ishizawa, K., Ichinohasama, R., Kameoka, J., Meguro, K., and Sasaki, T. (2004). Detection of clone-specific immunoglobulin heavy chain genes in the bone marrow of B-cell-lineage lymphoma after treatment. The Tohoku journal of experimental medicine 203, 155-164.
Hughesman, C. B., Lu, X. J. D., Liu, K. Y. P., Zhu, Y., Towle, R. M., Haynes, C., and Poh, C. F. (2017). Detection of clinically relevant copy number alterations in oral cancer progression using multiplexed droplet digital PCR. Scientific reports 7, 11855.
Lefranc, M. P. (2001). Nomenclature of the human immunoglobulin heavy (IGH) genes. Experimental and clinical immunogenetics 18, 100-116.
Linnebacher, M., and Maletzki, C. (2012). Tumor-infiltrating B cells: The ignored players in tumor immunology. Oncoimmunology 1, 1186-1188.
Linnemann, C., Mezzadra, R., and Schumacher, T. N. (2014). TCR repertoires of intratumoral T-cell subsets. Immunological reviews 257, 72-82.
Liu, Y. J., Malisan, F., de Bouteiller, O., Guret, C., Lebecque, S., Banchereau, J., Mills, F. C., Max, E. E., and Martinez-Valdez, H. (1996). Within germinal centers, isotype switching of immunoglobulin genes occurs after the onset of somatic mutation. Immunity 4, 241-250.
Lucio, P., Parreira, A., van den Beemd, M. W., van Lochem, E. G., van Wering, E. R., Baars, E., Porwit-MacDonald, A., Bjorklund, E., Gaipa, G., Biondi, A., et al. (1999). Flow cytometric analysis of normal B cell differentiation: a frame of reference for the detection of minimal residual disease in precursor-B-ALL. Leukemia 13, 419-427.
Ollila, J., and Vihinen, M. (2005). B cells. The international journal of biochemistry & cell biology 37, 518-523.
Pekin, D., Skhiri, Y., Baret, J. C., Le Corre, D., Mazutis, L., Salem, C. B., Millot, F., El Harrak, A., Hutchison, J. B., Larson, J. W., et al. (2011). Quantitative and sensitive detection of rare mutations using droplet-based microfluidics. Lab on a chip 11, 2156-2166.
Pichugin, A., larovaia, O. V., Gavrilov, A., Sklyar, I., Barinova, N., Barinov, A., lvashkin, E., Caron, G., Aoufouchi, S., Razin, S. V., et al. (2017). The IGH locus relocalizes to a “recombination compartment” in the perinucleolar region of differentiating B-lymphocytes. Oncotarget 8, 40079-40089.
Pohl, G., and Shih Ie, M. (2004). Principle and applications of digital PCR. Expert review of molecular diagnostics 4, 41-47.
Pongers-Willemse, M. J., Seriu, T., Stolz, F., d'Aniello, E., Gameiro, P., Pisa, P., Gonzalez, M., Bartram, C. R., Panzer-Grumayer, E. R., Biondi, A., et al. (1999). Primers and protocols for standardized detection of minimal residual disease in acute lymphoblastic leukemia using immunoglobulin and T cell receptor gene rearrangements and TALI deletions as PCR targets: report of the BIOMED-1 CONCERTED ACTION: investigation of minimal residual disease in acute leukemia. Leukemia 13, 110-118.
Radich, J., Ladne, P., and Gooley, T. (1995). Polymerase chain reaction-based detection of minimal residual disease in acute lymphoblastic leukemia predicts relapse after allogeneic BMT. Biology of blood and marrow transplantation: journal of the American Society for Blood and Marrow Transplantation 1, 24-31.
Robins, H. S., Ericson, N. G., Guenthoer, J., O'Briant, K. C., Tewari, M., Drescher, C. W., and Bielas, J. H. (2013). Digital genomic quantification of tumor-infiltrating lymphocytes. Sci Trans! Med 5, 214ra169.
Schatton, T., Scolyer, R. A., Thompson, J. F., and Mihm, M. C., Jr. (2014). Tumor-infiltrating lymphocytes and their significance in melanoma prognosis. Methods in molecular biology (Clifton, NJ) 1102, 287-324.
Schwartz, M., Zhang, Y., and Rosenblatt, J. D. (2016). B cell regulation of the anti-tumor response and role in carcinogenesis. J Immunother Cancer 4, 40.
Shen, M., Wang, J., and Ren, X. (2018). New Insights into Tumor-Infiltrating B Lymphocytes in Breast Cancer: Clinical Impacts and Regulatory Mechanisms. Frontiers in immunology 9, 470.
Szczepanski, T., Orfao, A., van der Velden, V. H., San Miguel, J. F., and van Dongen, J. J. (2001). Minimal residual disease in leukaemia patients. The Lancet Oncology 2, 409-417.
Tewhey, R., Warner, J. B., Nakano, M., Libby, B., Medkova, M., David, P. H., Kotsopoulos, S. K., Samuels, M. L., Hutchison, J. B., Larson, J. W., et al. (2009). Microdroplet-based PCR enrichment for large-scale targeted sequencing. Nat Biotechnol 27, 1025-1031.
Theurich, S., Schlaak, M., Steguweit, H., Heukamp, L. C., Wennhold, K., Kurschat, P., Rabenhorst, A., Hartmann, K., Schlosser, H., Shimabukuro-Vornhagen, A., et al. (2016). Targeting Tumor-Infiltrating B Cells in Cutaneous T-Cell Lymphoma. Journal of clinical oncology: official journal of the American Society of Clinical Oncology 34, e110-116.
Togashi, Y., Soda, M., Sakata, S., Sugawara, E., Hatano, S., Asaka, R., Nakajima, T., Mano, H., and Takeuchi, K. (2012). KLC1-ALK: a novel fusion in lung cancer identified using a formalin-fixed paraffin-embedded tissue only. PloS one 7, e31323.
van Dongen, J. J., Langerak, A. W., Bruggemann, M., Evans, P. A., Hummel, M., Lavender, F. L., Delabesse, E., Davi, F., Schuuring, E., Garcia-Sanz, R., et al. (2003). Design and standardization of PCR primers and protocols for detection of clonal immunoglobulin and T-cell receptor gene recombinations in suspect lymphoproliferations: report of the BIOMED-2 Concerted Action BMH4-CT98-3936. Leukemia 17, 2257-2317.
van Dongen, J. J., Seriu, T., Panzer-Grumayer, E. R., Biondi, A., Pongers-Willemse, M. J., Corral, L., Stolz, F., Schrappe, M., Masera, G., Kamps, W. A., et al. (1998). Prognostic value of minimal residual disease in acute lymphoblastic leukaemia in childhood. Lancet 352, 1731-1738.
Vettermann, C., and Schlissel, M. S. (2010). Allelic exclusion of immunoglobulin genes: models and mechanisms. Immunological reviews 237, 22-42.
Vogelstein, B., and Kinzler, K. W. (1999). Digital PCR. Proc Natl Acad Sci U S A 96, 9236-9241.
Walker, R. A. (2006). Quantification of immunohistochemistry--issues concerning methods, utility and semiquantitative assessment I. Histopathology 49, 406-410.
Wells, D. A., Benesch, M., Loken, M. R., Vallejo, C., Myerson, D., Leisenring, W. M., and Deeg, H. J. (2003). Myeloid and monocytic dyspoiesis as determined by flow cytometric scoring in myelodysplastic syndrome correlates with the IPSS and with outcome after hematopoietic stem cell transplantation. Blood 102, 394-403.
Wells, D. A., Sale, G. E., Shulman, H. M., Myerson, D., Bryant, E. M., Gooley, T., and Loken, M. R. (1998). Multidimensional flow cytometry of marrow can differentiate leukemic from normal lymphoblasts and myeloblasts after chemotherapy and bone marrow transplantation. Am J Clin Pathol 110, 84-94.
Whale, A. S., Huggett, J. F., and Tzonev, S. (2016). Fundamentals of multiplexing with digital PCR. Biomolecular detection and quantification 10, 15-23.
Wood, B., Jevremovic, D., Bene, M. C., Yan, M., Jacobs, P., and Litwin, V. (2013). Validation of cell-based fluorescence assays: practice guidelines from the ICSH and ICCS—part V—assay performance criteria. Cytometry B Clin Cytom 84, 315-323.
Zhong, Q., Bhattacharya, S., Kotsopoulos, S., Olson, J., Taly, V., Griffiths, A. D., Link, D. R., and Larson, J. W. (2011). Multiplex digital PCR: breaking the one target per color barrier of quantitative PCR. Lab on a chip 11, 2167-2174.
Zoutman, W. H., Nell, R. J., and van der Velden, P. A. (2019). Usage of Droplet Digital PCR (ddPCR) Assays for T Cell Quantification in Cancer. Methods Mol Biol 1884, 1-14.
Zoutman, W. H., Nell, R. J., Versluis, M., van Steenderen, D., Lalai, R. N., Out-Luiting, J. J., de Lange, M. J., Vermeer, M. H., Langerak, A. W., and van der Velden, P. A. (2017). Accurate Quantification of T Cells by Measuring Loss of Germline T-Cell Receptor Loci with Generic Single Duplex Droplet Digital PCR Assays. J Mol Diagn 19, 236-243.
Katz, D., et al., Obtaining confidence intervals for the risk ratio in cohort studies. Biometrics, 1978: p. 469-474.
Cancer Genome Atlas Research, N., et al., The Cancer Genome Atlas Pan-Cancer analysis project. Nat Genet, 2013. 45(10): p. 1113-20.
Taylor, A.M., et al., Genomic and Functional Approaches to Understanding Cancer Aneuploidy. Cancer Cell, 2018. 33(4): p. 676-689 e3.
Nareyeck, G., et al., Establishment and characterization of two uveal melanoma cell lines derived from tumors with loss of one chromosome 3. Exp Eye Res, 2006. 83(4): p. 858-64.Fatima Zare et al. BMC Bioinformatics. 2017 May 31; 18(1):286

TABLE 1

| | | Genomic | | | |
|---|---|---|---|---|---|
| Assay | Gene | feature | Forward primer (5″ to 3′) | Reverse primer (5″ to 3′) | Probe (5″ to 3′) |
| ΔD | TRD | Dδ2-Dδ2 | GCTGGCTGTAATGGGAATGT | TAATGGCTTGATAAAGATAAGTGATCAT | TGTGAAGATGTCTGTAGCCATCTTAT |
| | | (intergenic) | (SEQ ID NO: 1) | (SEQ ID NO: 8) | (SEQ ID NO: 15) |
| ΔB | TRB | Dβ2-Dβ2 | GCCATGCACTTTCCCTTTCG | ACAGAGTCCATCCACAGGG | TGGACCCTCACAGAGGGAGCA |
| | | (intergenic) | (SEQ ID NO: 2) | (SEQ ID NO: 9) | (SEQ ID NO: 16) |
| Regional | TRB | TRBC2 | CCACAGGTCAAGAGAAAGGA | TCCTGGGTGAGGATGAAGAA (SEQ ID | GGCTAGCTCCAAAACCATCCCAGGTCA |
| Corrector | | (coding) | (SEQ ID NO: 3) | NO: 10) | (SEQ ID NO: 17) |
| for ΔB | | | | | |
| ΔH | IGH@ | IGHD7-27- | AGGGTTTTGGCTGAGCTG | TGGTTTTTGTAGAGCTGCCA | ACCACTGTGCTAACTGGGGACACAGTG |
| | | IGHJ1 | (SEQ ID NO: 4) | (SEQ ID NO: 11) | (SEQ ID NO: 18) |
| | | (intergenic) | | | |
| ΔS | IGH@ | IGHD | CTTTCTGCTCTCTGGTAGCC | GGCTGTCTTTCAGGTGAAGT (SEQ ID | GGGCGTCCTGCTCTTCTGGGGC (SEQ |
| | | (coding) | (SEQ ID NO: 5) | NO: 12) | ID NO: 19) |
| Regional | IGH@ | IGHA2 | GCTGAGCCTCGACAGCAC | GGTCACACTGAGTGGCTCCT (SEQ ID | CCCCAAGATGGGAACGTGGTC (SEQ ID |
| Corrector | | (coding) | (SEQ ID NO: 6) | NO: 13) | NO: 20) |
| for ΔH | | | | | |
| and ΔS | | | | | |
| Regional | TMEM121 | (coding) | CCTGCACCTTCCTGGAGTA | GCAGCTCCAGTGATAGCG (SEQ ID | TGCGCGACTTCCCGCCGC (SEQ ID NO: |
| Corrector | | | (SEQ ID NO: 7) | NO: 14) | 21) |
| for ΔH | | | | | |
| and ΔS | | | | | |

TABLE 2

| | | Genomic | Forward primer | Reverse primer | | |
|---|---|---|---|---|---|---|
| Assay | Gene | feature | (5″ to 3′) | (5″ to 3′) | Probe (5″ to 3′) | Remark |
| ΔS | IGH@ | IGHM | CTGGGCCCATATGCT | TGATGAAGGCAGCAGA | TGCTCACCGGTGGGTGT | Alternative |
| (alternative) | | | TAGTC | TAGC | GGGG | ΔS marker |
| | | | (SEQ ID NO: 22) | (SEQ ID NO: 24) | (SEQ ID NO: 26) | |
| Regional | IGH@ | IGHA2 | AGCCTCGACAGCACC | GGTCACACTGAGTGGC | CGTGGTCGTCGCATGCC | Optimised |
| Corrector for | | | CC | TCCT | (SEQ ID NO: 27) | assay for |
| ΔH and ΔS | | | (SE ID NO: 23) | (SEQ ID NO: 25) | | improved CD |
| (optimised) | | | | | | dPCR |
| | | | | | | separation |
| | | | | | | of positive |
| | | | | | | droplets |

1. A method for determining the VDJ rearranged human T-cell fraction in a sample, the method comprising:
a) quantifying, in the sample, the amount of:
a diploid reference DNA marker;
a TCR DNA marker selected from an intergenic region between D62 and D63 on chromosome 14q11.2 or an intergenic region between D61 and 161.1 on chromosome 7q34; and
a DNA regional corrector of the TCR marker; and
b) determining the VDJ rearranged human T-cell fraction in the sample based on the quantification obtained in step a), wherein the VDJ rearranged T-cell fraction is determined as:
T-cell fraction=([DNA regional corrector]−[TCR DNA marker])/[diploid reference DNA marker].

2. The method of claim 1, wherein the VDJ rearranged human T-cells express a T-cell receptor.

3. The method of claim 1, wherein the TCR DNA marker is an intergenic region between D62 and D63 on chromosome 14q11.2 and the DNA regional corrector is selected from the group consisting of: CHD8, METTL3, SALL2 and TOX4.

4. The method of claim 1, wherein the TCR DNA marker is an intergenic region between Dβ1 and Jβ1.1 on chromosome 7q34 and the DNA regional corrector is selected from the group consisting of: TRBC2, BRAF, MOXD2P, PRSS58, MGAM, TAS2R38, and CLEC5A.

5. The method of claim 1, wherein the diploid reference DNA marker is selected from the group consisting of: exon 14 of DNM3, TTC5, TERT, VOPP1.

6. The method of claim 1, wherein the sample comprises malignant cells and/or cells with DNA copy number instability, or the sample originates from malignant cells and/or originates from cells with DNA copy number instability.

7. The method of claim 1, wherein the sample comprises DNA having copy number alterations of chromosome 14q or chromosome 7q.

8. The method of claim 1, wherein the sample is a tissue sample or a body fluid sample, optionally wherein the body fluid sample is vitreous fluid, cerebrospinal fluid, peritoneal fluid, amniotic fluid, pleural fluid or synovial fluid.

9. The method of claim 1, wherein the diploid reference DNA marker, TCR DNA marker and DNA regional corrector are quantified using a multiplex assay.

10. The method of claim 1, wherein the diploid reference DNA marker, TCR DNA marker and regional corrector are quantified by digital PCR.

11. The method of claim 1, wherein the sample is obtained from a subject.

12. The method of claim 1, wherein the method is for monitoring disease progression, determining the effect of a medicament used in the treatment of a disease, determining disease prognosis, or diagnosing a disease.

13. The method of claim 12, wherein the disease is an infectious disease, an autoimmune disease or a cancer.

14. The method of claim 13, wherein the cancer is uveal melanoma, skin melanoma or any other solid tumour.

15. The method of claim 13, wherein the autoimmune disease is rheumatoid arthritis, multiple sclerosis, type 1 diabetes or inflammatory bowel disease.

16. The method of claim 13, wherein the infectious disease is:
(i) a viral infection, optionally wherein the viral infection is HIV or hepatitis; or
(ii) a bacterial infection, optionally wherein the bacterial infection is tuberculosis or pertussis.

17. A method for determining the VDJ rearranged human B-cell fraction in a sample, the method comprising:
a) quantifying, in the sample, the amount of:
a diploid reference DNA marker; and
a B-cell DNA marker comprising an intergenic sequence between IGHD7-27 and lain at chromosome 14q32.33; and
b) determining the VDJ rearranged human B-cell fraction in the sample based on the quantification obtained in step a), wherein the VDJ rearranged human B-cell fraction is determined as:
B-cell fraction=1−([B-cell DNA marker]/[diploid reference DNA marker]).

18. A method for determining the VDJ rearranged human B-cell fraction in a sample, the method comprising:
a) quantifying, in the sample, the amount of:
a diploid reference DNA marker;
a B-cell DNA marker comprising an intergenic sequence between IGHD7-27 and lain at chromosome 14q32.33; and
a DNA regional corrector of the B-cell DNA marker;
b) determining the VDJ rearranged human B-cell fraction in the sample based on the quantification obtained in step a),
wherein the VDJ rearranged human B-cell fraction is determined as:
B-cell fraction=([DNA regional corrector]−[B-cell DNA marker])/[diploid reference DNA marker].

19. The method of claim 18, wherein the regional corrector is selected from the group consisting of: IGHA2, TMEM121, MARK3, BAG5, KLC1, MTA1, CRIP2, PACS2, BRF1, JAG2 and PLD4.

20. The method of claim 17, wherein step a) of the method further comprises determining the class-switched VDJ rearranged human B-cell fraction in the sample by:
i) quantifying, in the sample, a class-switched B-cell DNA marker comprising a sequence of IGHD at chromosome 14q32.33; and
ii) determining the class-switched VDJ rearranged human B-cell fraction.

21. A method for determining the class-switched human B-cell fraction in a sample, the method comprising:
a) quantifying, in the sample, the amount of:
a diploid reference DNA marker; and
a class-switched B-cell DNA marker comprising a sequence of IGHD at chromosome 14q32.33; and
b) determining the class-switched human B-cell fraction in the sample based on the quantification obtained in step a), wherein the class-switched human B-cell fraction is determined as:
class-switched fraction=1−([class-switched B-cell DNA marker]/[diploid reference DNA marker]).

22. The method of claim 21, wherein the class-switched human B-cell fraction is determined as:
class-switched fraction={1−([class-switched B-cell DNA marker]/[diploid reference DNA marker])}/allelic factor for the class-switched B-cell DNA marker.

23. A method for determining the class-switched human B-cell fraction in a sample, the method comprising:
a) quantifying, in the sample, the amount of:
a diploid reference DNA marker;
a class-switched B-cell DNA marker comprising a sequence of IGHD at chromosome 14q32.33; and
a DNA regional corrector of the class-switched B-cell DNA marker; and
b) determining the class-switched human B-cell fraction in the sample based on the quantification obtained in step a), wherein the class-switched human B-cell fraction is determined as:
class-switched fraction=([DNA regional corrector]−[class-switched B-cell DNA marker])/[diploid reference DNA marker].

24. The method of claim 23, wherein the class-switched human B-cell fraction is determined as:
class-switched fraction={([DNA regional corrector]−[class-switched B-cell DNA marker])/[diploid reference DNA marker]}/allelic factor for the class-switched B-cell DNA marker.

25. The method of claim 23, wherein the regional corrector is selected from the group consisting of: IGHA2, TMEM121, MARK3, BAG5, KLC1, MTA1, CRIP2, PACS2, BRF1, JAG2 and PLD4.

26. The method of claim 17, wherein the VDJ rearranged human B-cells express a B-cell receptor or an antibody.

27. The method of claim 17, wherein the diploid reference DNA marker is selected from the group consisting of: exon 14 of DNM3, TTC5, TERT, VOPP1.

28. The method of claim 17, wherein the sample comprises malignant cells and/or cells with DNA copy number instability, or the sample originates from malignant cells and/or originates from cells with DNA copy number instability.

29. The method of claim 17, wherein the sample comprises DNA having copy number alterations of chromosome 14q.

30. The method of claim 17, wherein the sample is a tissue sample or a body fluid sample, optionally wherein the body fluid sample is vitreous fluid, cerebrospinal fluid, peritoneal fluid, amniotic fluid, pleural fluid or synovial fluid.

31. The method of claim 17, wherein the diploid reference DNA marker, B-cell DNA marker and optionally the DNA regional corrector are quantified using a multiplex assay.

32. The method of claim 17, wherein the diploid reference DNA marker, B-cell DNA marker and optionally the DNA regional corrector are quantified by digital PCR.

33. The method of claim 17, wherein the sample is obtained from a subject.

34. The method of claim 17, wherein the method is for monitoring disease progression, or determining the effect of a medicament used in the treatment of a disease, or determining disease prognosis, or diagnosing a disease.

35. The method of claim 34, wherein the disease is selected from an infectious disease, an autoimmune disease or a cancer.

36. The method of claim 35, wherein the cancer is a B-cell lymphoma or any solid tumour that becomes inflamed, optionally wherein the solid tumour is melanoma.

37. The method of claim 35, wherein the autoimmune disease is rheumatoid arthritis, multiple sclerosis, type 1 diabetes or inflammatory bowel disease.

38. The method of claim 35, wherein the infectious disease is:
(i) a viral infection, optionally wherein the viral infection is hepatitis; or
(ii) a bacterial infection, optionally wherein the bacterial infection is tuberculosis or pertussis.

39. (canceled)

40. (canceled)

41. (canceled)

42. (canceled)

43. (canceled)

44. (canceled)

45. (canceled)

46. (canceled)


SEQUENCE LISTING (27 sequences)

SEQ ID NO: 1 - DNA, 20 bp, Artificial Sequence
  forward primer for TRD
gctggctgtaatgggaatgt

SEQ ID NO: 2 - DNA, 20 bp, Artificial Sequence
  forward primer for TRB
gccatgcactttccctttcg

SEQ ID NO: 3 - DNA, 20 bp, Artificial Sequence
  forward primer for TRB
ccacaggtcaagagaaagga

SEQ ID NO: 4 - DNA, 18 bp, Artificial Sequence
  forward primer for IGH@
agggttttggctgagctg

SEQ ID NO: 5 - DNA, 20 bp, Artificial Sequence
  forward primer for IGH@
ctttctgctctctggtagcc

SEQ ID NO: 6 - DNA, 18 bp, Artificial Sequence
  forward primer for IGH@
gctgagcctcgacagcac

SEQ ID NO: 7 - DNA, 19 bp, Artificial Sequence
  forward primer for TMEM121
cctgcaccttcctggagta

SEQ ID NO: 8 - DNA, 28 bp, Artificial Sequence
  reverse primer for TRD
taatggcttgataaagataagtgatcat

SEQ ID NO: 9 - DNA, 19 bp, Artificial Sequence
  reverse primer for TRB
acagagtccatccacaggg

SEQ ID NO: 10 - DNA, 20 bp, Artificial Sequence
  reverse primer for TRB
tcctgggtgaggatgaagaa

SEQ ID NO: 11 - DNA, 20 bp, Artificial Sequence
  reverse primer for IGH@
tggtttttgtagagctgcca

SEQ ID NO: 12 - DNA, 20 bp, Artificial Sequence
  reverse primer for IGH@
ggctgtctttcaggtgaagt

SEQ ID NO: 13 - DNA, 20 bp, Artificial Sequence
  reverse primer for IGH@
ggtcacactgagtggctcct

SEQ ID NO: 14 - DNA, 18 bp, Artificial Sequence
  reverse primer for TMEM121
gcagctccagtgatagcg

SEQ ID NO: 15 - DNA, 26 bp, Artificial Sequence
  probe for TRD
tgtgaagatgtctgtagccatcttat

SEQ ID NO: 16 - DNA, 21 bp, Artificial Sequence
  probe for TRB
tggaccctcacagagggagca

SEQ ID NO: 17 - DNA, 27 bp, Artificial Sequence
  probe for TRB
ggctagctccaaaaccatcccaggtca

SEQ ID NO: 18 - DNA, 27 bp, Artificial Sequence
  probe for IGH@
accactgtgctaactggggacacagtg

SEQ ID NO: 19 - DNA, 22 bp, Artificial Sequence
  probe for IGH@
gggcgtcctgctcttctggggc

SEQ ID NO: 20 - DNA, 21 bp, Artificial Sequence
  probe for IGH@
ccccaagatgggaacgtggtc

SEQ ID NO: 21 - DNA, 18 bp, Artificial Sequence
  probe for TMEM121
tgcgcgacttcccgccgc

SEQ ID NO: 22 - DNA, 20 bp, Artificial Sequence
  forward primer for IGH@
ctgggcccatatgcttagtc

SEQ ID NO: 23 - DNA, 17 bp, Artificial Sequence
  forward primer for IGH@
agcctcgacagcacccc

SEQ ID NO: 24 - DNA, 20 bp, Artificial Sequence
  reverse primer for IGH@
tgatgaaggcagcagatagc

SEQ ID NO: 25 - DNA, 20 bp, Artificial Sequence
  reverse primer for IGH@
ggtcacactgagtggctcct

SEQ ID NO: 26 - DNA, 21 bp, Artificial Sequence
  probe for IGH@
tgctcaccggtgggtgtgggg

SEQ ID NO: 27 - DNA, 17 bp, Artificial Sequence
  probe for IGH@
cgtggtcgtcgcatgcc